(19) United States
(12) Patent Application Publication
(10) Pub. No.: US 2023/0210692 A1
(43) Pub. Date: Jul. 6, 2023

(54) Disposable phaco handpiece

(71) Applicant: H. P. Braem AG, Bronschhofen (CH)

(72) Inventors: Guido Casutt, Bronschhofen (CH); Nimrod Meier, Bronschhofen (CH)

(21) Appl. No.: 17/563,114

(22) Filed: Dec. 28, 2021

(51) Int. Cl. A61F 9/007

(52) U.S. Cl. CPC A61F 9/00745

(57) ABSTRACT

A disposable medical handpiece configured for phaco emulsification surgery includes an ultrasonic drive coupled to a phaco tip for vibrating the phaco tip at an ultrasonic frequency for emulsifying cataract. The phaco tip has an aspiration passage coupled to a cannula disposed within the ultrasonic drive for aspirating the emulsified cataract. The ultrasonic drive and the phaco tip are disposed in a housing having an integrated irrigation tube for irrigating the surgery site with an irrigation liquid. The disposable medical handpiece is configured to allow the ultrasonic drive to be reusable while the cannula and the housing to be disposable. The medical handpiece can include haptic feedback to allow the surgeon to know the status of the handpiece and the characteristics of the cataract. The medical handpiece can include a frequency selector to allow the surgeon to change the ultrasonic frequency based on the characteristics of the cataract.

The present invention relates to a surgical instrument in ophthalmology, such as a medical handpiece used to perform phacoemulsification (phaco) on patients with cataracts.

### BACKGROUND OF THE INVENTION

Phacoemulsification is a technique commonly used in modern eye cataract surgery. Using a phaco handpiece, e.g., a surgical instrument which can be used in cataract surgery, a surgeon can remove cataracts by making only a small incision into the eye. The phaco handpiece can be designed as a hollow needle, which is inserted through the incision to make contact with the cataracts. The needle is coupled to an ultrasonic transducer, which can cause the tip of the needle to vibrate at an ultrasonic frequency to break apart and emulsify the cataract. A rinsing fluid, such as a saline liquid, is supplied around the exterior of the hollow needle to maintain an ocular pressure in the eye. The liquid and the emulsified pieces of the cataracts are removed through an aspiration line, for example, through the pathway in the hollow needle.

Non-disposable phaco handpieces require rigorous cleaning and sterilization after each use to avoid any possible cross-contamination among patients, for example, due to the aspiration and irrigation pathways for fluids are contaminated with biological waste. The sterilization procedure can be costly, in term of labor, time and lifetime reduction of the handpiece, and as a result, can increase the cost of cataract surgery.

### SUMMARY OF THE EMBODIMENTS

The present invention relates to a surgical instrument in ophthalmology, such as handpiece used to perform phacoemulsification (phaco) on patients with cataracts. The surgical instrument can include an ultrasonic surgery unit, which can be used in cataract surgery.

In some embodiments, the present invention discloses a disposable phaco handpiece for phacoemulsification eye surgery, e.g., a phaco handpiece having disposable fluid pathways such as the aspiration and irrigation tubes. The disposable phaco handpiece can reduce cleaning time and effort, reduce cross-contamination, and increase the lifespan of the handpiece. Furthermore, the current invention allows different sonotrode extensions to be used to excite different motions at the tip of the handpiece.

### Components of a Phaco Handpiece

In some embodiments, the phaco handpiece can be a component-wise disposable device including components that can be disposable, reusable, or can be selected to be disposable or reusable.

The phaco handpiece can include an ultrasonic drive that includes an ultrasonic transducer and a sonotrode coupled to the ultrasonic transducer. The ultrasonic transducer can include a piezo crystal power unit to generate ultrasonic vibration, which can be transferred to the sonotrode. The ultrasonic drive can be the most expensive component of the phaco handpiece, and thus are designed with shielding from the contaminated fluid and also from the surgical ambient. The ultrasonic drive thus can be reused, e.g., removed from the used phaco handpiece to be optionally cleaned and sterilized before assembled in a new phaco handpiece.

The ultrasonic drive can include a cable to provide power to the ultrasonic transducer, for example, from a driver system. The cable can be hardwired to the ultrasonic transducer to be an integral part of the ultrasonic drive, or can be a separate component coupled to the ultrasonic transducer using a removable connector. The connector can also be shielded as part of the shielding for the ultrasonic drive, leaving the cable exposed to the surgical ambient. The cable can be disposed after a single use, or can be reused, since the cable is only exposed to the surgical ambient and not to the contaminated fluid. Alternatively, the cable can be shielded, such as being plastic wrapped, to allow the cable to be reused without or with minimum cleaning, for example, by replacing the plastic wrap.

The ultrasonic transducer and the sonotrode are hollow throughout to receive a cannula, which is an aspiration tube configured to provide an aspiration passage for the emulsified fluid to be removed from the surgery site. The cannula is configured to be removably inserted into the ultrasonic drive along an axis of the ultrasonic drive, e.g., the cannula can also be removed from the ultrasonic drive. The cannula can have seals at both ends, e.g., one end making a seal with the sonotrode and the other end making a seal with an aspiration pumping system. The seals can be configured to prevent contamination waste in the cannula from leaking away to the surrounding components. Seals of any type can be used, including a direct form fit without any sealing material, including the standard leak-free Luer connections for small-scale fluid fittings using male-taper fittings and mating female parts.

At the seal end with the sonotrode, pressure can be applied to the cannula by the phaco tip threading to the sonotrode and therefore squeezing the cannula against the sonotrode. At the seal end with the aspiration pumping system, the cannula can be protruded from the ultrasonic drive to allow a Luer type connecting piece to couple the cannula to an aspiration pumping system. The length of the connecting piece can be optimized to provide a leak-free seal with the cannula during the surgical operation, e.g., to remain a leak-free seal even with the various movements of the phaco handpiece.

The cannula can be configured to be single use, e.g., the cannula can be disposed after being used in an eye surgery since the contaminated waste is conducted through the inside of the cannula. Soft materials, e.g., having lower hardness than the sonotrode and the phaco tip, can be used to form the cannula. A cannula made from a softer material than the sonotrode and the phaco tip can have the additional advantage of not causing wear or damage to the sonotrode when forming the seal with the sonotrode, which can allow the sonotrode to be reused for multiple times. In some embodiments, hard materials can be used to form the cannula, e.g., as hard as the sonotrode material or harder than the sonotrode material.

The interface between the ultrasonic drive and the cannula can be configured to not disturb the vibration transfer, e.g., from the ultrasonic transducer to the sonotrode to the cannula. The friction between the two surfaces, e.g., the outer surface of the cannula and the inner surface of the sonotrode can be configured to minimize or to prevent the loss of vibration energy when transferring from the sonotrode to the cannula. For example, the surface roughness of the cannula and the sonotrode can be controlled, for example, to be less than Ra 50 microns, or less than Ra 20 or 10 microns, with Ra being the average surface roughness. In addition, the gap between the cannula and the sonotrode can be small to prevent loss of ultrasonic energy transfer, for example, the gap can be less than 100 microns, less than 50 microns, or less than 20 microns.

A phaco tip can be coupled to the cannula and also to the sonotrode. The phaco tip can be constructed in the form of a hollow needle with a small needle tip for ease of entering the small incision at the eye. The phaco tip is configured to be coupled to the cannula so that the hollow portion of the phaco tip connects with the cannula to form a fluid pathway for removing the emulsified fluid at the surgery site. The coupling of the phaco tip and the cannula can be leak-free, e.g., having a tight seal of any type, to prevent the contaminated waste in the fluid pathway to contact the ultrasonic drive to contaminate the ultrasonic drive. For example, the sealing can be a direct form fit with no sealing material, e.g., the phaco tip is pressed against the cannula to form the seal. Alternatively, a sealing element, such as a compressible material such as plastic or rubber, can be placed between the phaco tip and the cannula to form the seal. Pressure on the seal can be accomplished by the coupling between the phaco tip and the sonotrode.

In some embodiments, the phaco tip can be integrated with the cannula, such as bonding together to form a leak-free seal for the aspiration pathway from the tip of the phaco tip to the end of the cannula to the aspiration pumping system. The phaco tip and the cannula can be made from any material and can be mated by any procedure, such as using an adhesive.

The phaco tip can be configured to be coupled to the sonotrode, for example, to enclose the end portion of the sonotrode. The coupling of the phaco tip and the sonotrode can be leak-free, e.g., having a tight coupling of any type, to prevent the contaminated waste to contact the sonotrode or the ultrasonic transducer. The coupling of the phaco tip and the sonotrode can be a solid-to-solid coupling, e.g., without a thick vibration absorbance material, to present loss of ultrasonic vibration power transferred from the sonotrode to the phaco tip.

The coupling can be a threaded coupling, e.g., an inner thread at the phaco tip to be coupled with an outer thread at the sonotrode. By threading to the sonotrode, the phaco tip can exert a pressure to the cannula to form the leak-free seal. Other coupling can be used, such as a snap coupling, a magnetic coupling, or a press fit coupling.

The phaco tip can be configured to be single use or reusable. Since the phaco tip is in direct contact with the contaminated waste after each use, the phaco tip can be disposed after being used, to prevent cross contamination. Alternatively, since the phaco tip can be an expensive component of the phaco handpiece, the phaco tip can cleaned and sterilized after each use, to be re-assembled with other new or clean components of the phaco handpiece to form a new phaco handpiece.

The assembly of the phaco tip, the cannula, and the ultrasonic drive can be disposed in a housing. The housing can be configured to shield the ultrasonic drive from surgical fluid, such as from the irrigation liquid, by making a seal with the phaco tip. For example, the housing can be configured to cover the ultrasonic drive and a base portion of the phaco tip to isolate the ultrasonic drive from the irrigation liquid delivered to the tip portion of the phaco tip.

The housing can be configured to expose an end of the ultrasonic drive, e.g. the end having the protruded cannula and the cable connecting to the transducer. For example, the housing can include a small opening at one end for sealing with the base portion of the phaco tip, and a larger opening at the opposite end to allow the ultrasonic drive to be inserted to the housing in the order of the phaco tip, the sonotrode, and the ultrasonic transducer. The end of the ultrasonic transducer is inside the larger opening, and thus is exposed to the ambient.

Alternatively, the housing can include a housing cap or a housing end, which can be configured to cover the end of the housing, including a portion of the cannula connecting piece and a portion of the ultrasonic transducer cable. The covering of the cannula connecting piece and the cable can include a seal, such as having a compressible sealing material with openings for the cannula connecting piece and the cable to pass through while sealing the interior of the phaco handpiece at the openings.

The housing can be configured to not disturb the ultrasonic vibration transfer from the ultrasonic drive to the phaco tip. For example, the housing can contact the ultrasonic transducer of the ultrasonic drive for support, e.g., for coupling with the ultrasonic unit. Further, the housing can contact the ultrasonic transducer through a vibration damping material, such as a compressible material, to prevent a loss of vibration transfer. The housing can be configured to not contact the sonotrode, such as leaving a gap around the sonotrode, especially at the tip of the sonotrode to not interfere with the vibration of the sonotrode.

The housing can be integrated with an irrigation tube having an irrigation interface at one end of the housing, e.g., at the end of the housing having the larger opening. The irrigation interface can include a connecting piece for coupling the irrigation tube with an irrigation system. The coupling can include a leak-free coupling, such as a Luer type coupling. To avoid possible incorrect connections, the coupling to the irrigation interface require can a male Luer connector while the coupling to the cannula requires a female coupling. The irrigation tube can run along or under the surface of the housing to deliver the irrigation liquid from the irrigation interface to an opposite end of the housing, e.g., to the end of the housing having the small opening.

A phaco sleeve can be configured to be coupled with the small end of the housing, e.g., with the portion of the housing that makes a seal with the base portion of the phaco tip. The phaco sleeve can be supported by the housing, e.g., without contacting the phaco tip or the sonotrode. The phaco sleeve can be configured to cover the tip portion of the phaco tip, e.g., the portion of the phaco tip protruded from the housing seal. The phaco sleeve can be configured to interface with the irrigation tube, e.g., coupled to the irrigation tube to receive the irrigation liquid. Thus, the irrigation tube can be formed along or under a surface of the housing, having one end of the irrigation tube configured to be coupled to the irrigation system, and having an opposite end having an interface with the phaco sleeve.

The phaco sleeve can have one or more exit ports at sides of the sleeve at or near the tip of the phaco tip to deliver the irrigation liquid to the surgery site. Thus, the irrigation liquid can run from the irrigation system to the irrigation tube (through the irrigation interface), to the phaco sleeve (through the sleeve interface), and then exit through the exit ports. The sleeve can be sealed off at the tip of the phaco tip, to ensure that the irrigation liquid exits through the exit ports at the sides of the phaco sleeve.

The housing and the phaco sleeve can be configured to be disposable, e.g., single use. Soft materials can be used to form the housing and the phaco sleeve. An advantage of a soft housing is the low transfer of vibration energy, thus reducing potential vibration loss generated from the ultrasonic drive.

### Assembling of a Phaco Handpiece

For assembly, a new cannula is inserted to the hollow passage of a new or reprocessed ultrasonic drive from the sonotrode end. The cannula is completely inserted so that one end is in contact with the end of the sonotrode. The contact can be adjusted so that the cannula can make a seal with the sonotrode.

A new or reconditioned, e.g., cleaned and sterilized, phaco tip can be coupled to the sonotrode, fixing the cannula in place. For example, the phaco tip can have an inner thread, which can be screwed to the outer thread end of the sonotrode. The interface of the phaco tip to the cannula can be configured to make a seal by pressing on the cannula through the threaded coupling between the phaco tip and the sonotrode. Alternatively, other coupling mechanisms between the phaco tip and the sonotrode can be used, such as a snap fit in which the sonotrode is inserted into the opening at the end of the phaco tip to form a seal.

The ultrasonic drive can be equipped with a cable having a connector at the end to be electrically connected to a drive system. The cable can be an integral part of the ultrasonic drive, e.g., the cable can be firmly fixed to the ultrasonic drive. Alternatively, the ultrasonic drive can be configured to be coupled with a removable cable. e.g., the cable can have connectors at both ends. One connector end of the cable can be coupled to the ultrasonic transducer. The other connector end can be coupled to the driver system, for example, at a later time when the phaco handpiece is already fully assembled. Other assembling order can be used, such as the cable is coupled to the ultrasonic transducer at any time during the assembling process, such as at the beginning, to form an ultrasonic drive completed with the connecting cable.

A new housing having an integrated irrigation tube is then used to cover the assembly of the phaco tip and the ultrasonic drive with the cannula and the cable. The housing can be in the form of a tube, ergonomically designed for the surgeon hand. The housing can have a small opening at one end, and a larger opening at an opposite end. The ultrasonic drive assembly, including the phaco tip, the sonotrode, the ultrasonic transducer, and the cable, can be inserted through the larger opening so that the base portion of the phaco tip is protruded from the small opening. The small opening can be configured to seal on the base portion of the phaco tip, thus the ultrasonic drive is sealed from the ambient at the small opening end. The housing can contact the ultrasonic drive at the ultrasonic transducer, such as through a vibration damping material, to use the ultrasonic drive as a support for the housing while not disturb the vibration transfer from the power drive to the sonotrode to the phaco tip.

In some embodiments, the larger opening end can be open, e.g., exposing the end portion of the ultrasonic drive, the cable, and the cannula connecting piece. In some embodiments, a separate end cap, e.g., a housing end, can be configured to secure to the housing and to cover the ultrasonic drive and portions of the cable and the cannula connecting piece. The cable and the cannula connecting piece can be assembled as pass-through seals, e.g., the cable and the connecting piece are protruded from the housing end while being sealed from the interior of the housing.

A new phaco sleeve can then slide around the phaco tip to be coupled with the small end of the housing, e.g., the small end is the end making a seal with the protruded phaco tip. The phaco sleeve can be configured to cover the protruded phaco tip, with the tip end of the phaco tip protruded from the phaco sleeve. The phaco sleeve can have a fluid passage, which is configured to be coupled with the irrigation tube from the housing to receive the irrigation liquid. The phaco sleeve can have an exit port at a side of the phaco sleeve to deliver the irrigation liquid received from the irrigation tube to an area near the phaco tip. The phaco sleeve can surround the tip of the phaco tip without any gap, or with a small gap, e.g., much smaller than the exit port, to direct the irrigation liquid to exit through the exit port.

After the completion of the assembly, the phaco handpiece can be coupled to the driver system through the cable, to the aspiration pumping system through the cannula connecting piece, and to the irrigation system through the irrigation interface on the housing.

### Novelty

### A Cannula for Sealing the Ultrasonic Drive From the Aspiration Fluid

In some embodiments, the present invention discloses a phaco handpiece having reusable and disposable parts. The reusable parts can include the ultrasonic drive since the ultrasonic drive is sealed from the aspiration fluid with the cannula. By providing a cannula for containing the aspiration fluid along the ultrasonic drive, e.g., from the phaco tip to the connection piece for the aspiration system, the ultrasonic drive is not exposed to the aspiration fluid, and thus can be reused without or with minimum cleaning.

The cannula can be designed to achieve an effective vibration transfer with the ultrasonic drive by a small gap between the cannula and the sonotrode, together with an optimum friction between the cannula and the sonotrode. The friction can be changed by the cannula or the sonotrode having a low friction surface structure, such as made from a low friction material.

In some embodiments, the phaco handpiece can include a hard material sonotrode coupled with a softer material cannula. Alternatively, the cannula can include a hard material, such as harder than the sonotrode material or as hard as the sonotrode material. The cannula can be configured to be a disposable cannula, e.g., as a one-time-use component in the phaco handpiece.

In some embodiments, the coupling between the cannula and the sonotrode can include a direct form fit with no additional sealing material. The direct form fit can be in the form of a flange at the cannula end to be mated with a recess at the sonotrode. A force can be applied to the cannula flange from the coupling of the phaco tip with the sonotrode to enable the seal.

### Phaco Tip With Inner Thread and Direct Form Fit

In some embodiments, the phaco handpiece can include a phaco tip configured to form a seal with both the sonotrode and the cannula disposed within the sonotrode. The phaco tip can include a needle tip configured to enter the incision made at the eye. The phaco tip can also include a hollow passage passing from the needle tip to the opposite end of the phaco tip. The hollow passage can be configured to make a first seal with the cannula, to allow the fluid from the surgery site to be removed to an aspiration pumping system. The phaco tip can also include a second seal with the sonotrode, which is configured to prevent contamination of the sonotrode.

The phaco tip thus can include a direct form fit seal with the cannula end around the hollow passage in the phaco tip. The phaco tip can also include another seal with the sonotrode, such as a threaded seal, a snap seal, a press fit seal, or a magnetic seal. The seal with the sonotrode can be configured to provide a force acting on the form fit seal of the phaco tip with the cannula, which can provide a pressure on the form fit seal to maintain the seal structure.

### On-Off Haptic Feedback

In some embodiments, the phaco handpiece can include an on-off haptic feedback to provide indications on whether the phaco handpiece is on or off. The haptic feedback can include a sensor, such as a vibration coupler, such as a partial metal ring, coupled to the sonotrode. The haptic feedback can include a receiver, such as a feedback indicator, such as a metal plate, on an outer surface of the housing, e.g., at a location contacting the surgeon hand, to provide a haptic feedback to the surgeon. A solid connection can be provided between the sensor and the receiver, which can allow the vibration to reach the receiver when the sensor senses vibration at the sonotrode. The solid connection can be configured to optimize the haptic feedback, e.g., to provide a vibration at the receiver with an appropriate amplitude comfortable to the surgeon. Alternatively, an adjustable connection can be provided to allow the surgeon to change the vibration levels of the haptic feedback.

In operation, when the phaco handpiece is turned on, e.g., when the ultrasonic drive receives power from the driver system to generate the vibration at the sonotrode, the sensor can also be vibrated. The vibration from the sensor is communicated to the receiver, through the solid connection. Upon feeling the vibration in the hand, the surgeon can recognize that the phaco handpiece is on, e.g., there are ultrasonic vibrations at the phaco tip. When the phaco handpiece is turned off, there is no vibration at the sonotrode, which leads to no vibration at the receiver. The surgeon can recognize that the phaco handpiece is off.

The haptic feedback can be particular useful for a phaco handpiece having a single use plastic housing, since the plastic housing might not conduct vibration transfer well. In conjunction with a plastic housing, the haptic feedback can simulate the vibration effect of a metal housing while minimizing the loss of vibration energy to the housing.

Alternatively, the haptic feedback can include a vibration unit coupled to an outer surface of the housing. Power to the vibration unit can be provided by the driver system when the ultrasonic is turned on. For example, the vibration unit can receive power concurrently as the ultrasonic drive, thus, then the ultrasonic drive is turned on, the vibration unit also vibrates, which gives the surgeon a haptic feedback indicating that the ultrasonic drive is on. The power of the vibration unit can be adjustable, e.g., a strong vibration or a weak vibration, depending on a preference of the surgeon, for example, by adjusting the level of power applied to the vibration unit. An advantage of the haptic feedback using the vibration unit is the decoupling of the ultrasonic drive with the haptic feedback. The haptic feedback using the vibration unit does not disturb the ultrasonic vibration transfer at the sonotrode.

### Cataract Characteristic Haptic Feedback

In some embodiments, the phaco handpiece can include a material haptic feedback to provide information about the structure of the eye, such as the force to get into the eye lens and the information about the cataract, such as how hard or how soft the cataract is.

The haptic feedback can include an ultrasonic sensor configured to measure the ultrasonic disturbance on the ambient caused by the ultrasonic drive. The ultrasonic sensor can senses an ultrasonic field generated through the vibrations at the phaco tip and travelled through the ambient between the phaco tip and the ultrasonic sensor.

The measurement data from the ultrasonic sensor can be provided to a controller that can process the data to generate a parameter, such as hardness or softness, of the ambient surroundings of the phaco tip at the surgery site. The measurement data can be calibrated, for example, by calculating the field difference with and without the fluidic ambient at the surgery site.

A vibration unit can be coupled to an outer surface of the housing and is configured to receive information from the controller. Depending on the data processed from the ultrasonic sensor, the vibration unit can vibrate at different vibration amplitudes or at different pulse sequences, to let the surgeon know about the structure and characteristics of the cataract.

Alternatively, the haptic feedback can further include an additional ultrasonic drive configured to generate a different ultrasonic field. The different ultrasonic field can be selected to provide better information on the structure and characteristics of the fluid ambient at the surgery site, such as using a diagnostic ultrasonic frequency.

Alternatively, the haptic feedback can include a force sensor, such as a force transducer, coupled to the sonotrode to measure a force on the phaco tip. The force data can be supplied to the vibration unit to provide a haptic feedback to the surgeon.

### Multiple Frequency Phaco Handpiece

In some embodiments, the phaco handpiece can be configured to vibrate at different ultrasonic frequencies. The different frequencies can be selected based on the structure and characteristics of the cataract, which can be measured by a material haptic feedback. The ultrasonic transducer can include multiple oscillation circuits that generate ultrasonic vibrations at different frequencies. In addition, a selector can be included at the phaco handpiece to allow the surgeon to switch frequencies during the surgical operation.

### BRIEF DESCRIPTION OF THE DRAWINGS

FIG. 1 illustrates a schematic configuration of a phaco handpiece according to some embodiments.

FIG. 2 illustrates a perspective view of a phaco handpiece according to some embodiments.

FIGS. 3A - 3B illustrates flow charts for operations of a phaco handpiece according to some embodiments.

FIG. 4 illustrates an exploded view of a phaco handpiece according to some embodiments.

FIGS. 5A - 5E illustrate an assembling process for a phaco handpiece according to some embodiments.

FIG. 6 illustrates an assembling process for a phaco handpiece according to some embodiments.

FIG. 7 illustrates a disassembling process for a phaco handpiece according to some embodiments.

FIG. 8 illustrates an effective energy transfer with a small gap between a cannula and an ultrasonic drive according to some embodiments.

FIG. 9 illustrates an effective energy transfer with a small gap between a cannula and an ultrasonic drive according to some embodiments.

FIGS. 10A - 10D illustrate seal configurations between a cannula and a sonotrode according to some embodiments.

FIGS. 11A - 11B illustrate other seal configurations between a cannula and a sonotrode according to some embodiments.

FIGS. 12A - 12C illustrate flow charts for coupling a cannula, e.g., an aspiration tube, with an ultrasonic drive according to some embodiments.

FIG. 13 illustrates a flow chart for forming a seal between a cannula, e.g., an aspiration tube, and an ultrasonic drive according to some embodiments.

FIGS. 14A - 14B illustrate configurations for seal structures between a phaco tip and a cannula according to some embodiments.

FIGS. 15A - 15C illustrate a configuration for an integrated phaco tip and cannula according to some embodiments.

FIGS. 16A - 16C illustrate flow charts for forming a seal between a phaco tip and a cannula according to some embodiments.

FIGS. 17A - 17C illustrate a threaded coupling between a phaco tip and a sonotrode according to some embodiments.

FIGS. 18A - 18B illustrate other coupling configurations between a phaco tip and a sonotrode according to some embodiments.

FIGS. 19A - 19B illustrate flow charts for forming a phaco tip to make a seal with a cannula and a sonotrode according to some embodiments.

FIGS. 20A - 20C illustrate a configuration of a housing for a phaco handpiece according to some embodiments.

FIG. 20C1 - 20C4 show details of the housing assembly at portions 1 - 4.

FIGS. 21A - 21C illustrate a phaco sleeve coupled to a housing according to some embodiments.

FIGS. 22A - 22C illustrate a housing end for a housing according to some embodiments.

FIGS. 23A - 23B illustrate flow charts for forming elements of a phaco handpiece according to some embodiments.

FIG. 24 illustrates a flow chart for assembling a phaco handpiece according to some embodiments.

FIGS. 25A - 25F illustrate haptic feedback configurations for a phaco handpiece according to some embodiments.

FIGS. 26A - 26B illustrate another haptic feedback configuration for a phaco handpiece according to some embodiments.

FIGS. 27A - 27B illustrate flow charts for forming a phaco handpiece having an on-off haptic feedback according to some embodiments.

FIGS. 28A - 28B illustrate flow charts for operating a phaco handpiece having an on-off haptic feedback according to some embodiments.

FIGS. 29A - 29D illustrate a phaco handpiece having an ultrasonic haptic feedback according to some embodiments.

FIGS. 30A - 30D illustrate a phaco handpiece having a force transducer haptic feedback according to some embodiments.

FIGS. 31A - 31B illustrate flow charts for forming a phaco handpiece having a haptic feedback according to some embodiments.

FIGS. 32A - 32B illustrate flow charts for operating a phaco handpiece having a haptic feedback according to some embodiments.

FIGS. 33A - 33B illustrate a phaco handpiece configured for multiple ultrasonic frequencies according to some embodiments.

FIGS. 34A - 34B illustrate flow charts for forming a phaco handpiece having multiple frequencies according to some embodiments.

FIGS. 35A - 35D illustrate flow charts for operating a phaco handpiece having multiple frequencies according to some embodiments.

FIGS. 36A - 36C illustrate configurations of phaco handpieces having multiple haptic feedbacks with a frequency controller according to some embodiments.

### DETAILED DESCRIPTION OF THE EMBODIMENTS

In some embodiments, the present invention discloses a phaco handpiece for phacoemulsification surgery, such as to remove cataracts from the eyes of patients. Cataract is a clouding or loss of transparency of the lens of the eye or of its surrounding transparent membrane, which can obstruct the passage of light. Cataract can be caused by tissue breakdown and protein clumping. Cataract can be treated by a cataract surgery in which the cloudy natural lens is removed and replaced with an artificial lens.

During the cataract surgery, the tip end of the phaco handpiece is inserted into the eye to transmit energy generated by an ultrasonic transducer in the handpiece into the cataract tissue and fluid around the tip. The ultrasonic energy can emulsify the cataract tissue, and the emulsified fluid can be removed through an aspiration passage in the phaco tip. An irrigation tube in the handpiece can be configured to supply an irrigation liquid to the phaco tip to equalize the pressure in the eye.

The phaco handpiece can be configured to be a component-wise disposable phaco handpiece, e.g., the phaco handpiece can be designed to not require complete sterilization, for example, to provide a phaco handpiece having removable, disposable or limited use components carrying the fluid pathways, together with other components that are potentially exposed to the contaminated fluids. The disposable components of the phaco handpiece can be disposed after each surgical procedure, thus reducing cleaning time and effort, reduce cross-contamination, and increase the lifespan of the handpiece. Furthermore, the phaco handpiece can be incorporated with haptic feedback, which can provide indications to the surgeon about the operating status of the handpiece and the characteristics of the cataract.

### Components of a Phaco Handpiece

In some embodiments, the phaco handpiece can be a component-wise disposable device including components that can be disposable, reusable, or can be selected to be disposable or reusable. Components of the phaco handpiece that can come in contact with a fluid at a surgery site can be disposable. Components of the phaco handpiece that are expensive and do not come in contact with the fluid at the surgery site, can be designed with adequate shielding, e.g., designed with a disposable protective cover, to allow the expensive components to be reusable. In addition, the phaco handpiece can have some components that may be either disposable after a single use or can be reused after cleaning.

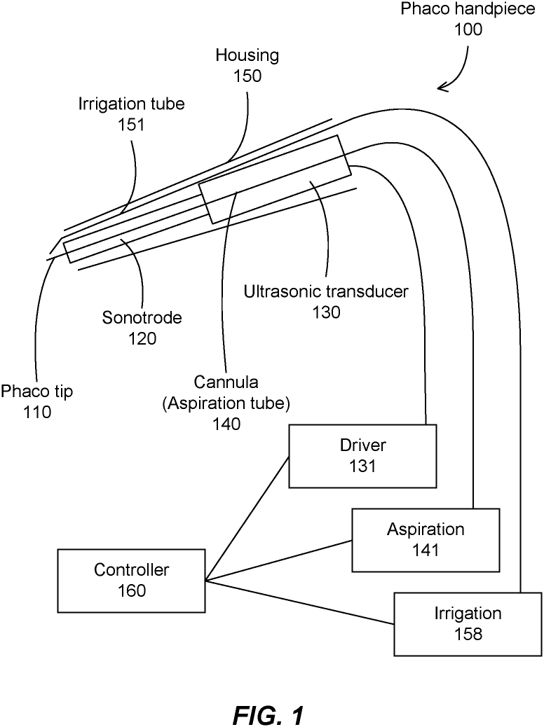

FIG. 1 illustrates a schematic configuration of a phaco handpiece according to some embodiments. A phaco handpiece 100 can include an ultrasonic drive having an ultrasonic transducer 130 coupled to a sonotrode 120, which is an ultrasonic horn or probe used for augmenting the oscillation displacement provided by the ultrasonic transducer 130. The ultrasonic transducer 130 can be powered by a driver system 131, such as through a power cable carrying electrical control signals to the ultrasonic transducer at the back end of the phaco handpiece. The ultrasonic vibrations transmitted to the sonotrode 120 can be transmitted to the tip of a phaco tip 110, so that the vibrations of the tip can be used to comminute cataract materials at the surgery site. The phaco tip 110 can have an aspiration passage connected to a cannula, e.g., an aspiration tube 140. The cannula can be coupled to an aspiration pumping system 141 to allow liquid and cataract fragments emulsified by the ultrasonic vibrations of the phaco tip to be aspirated away from the surgery site. The aspiration pumping system can include volumetric flow or positive displacement pumps, such as peristaltic or scroll pumps, or vacuum-based pumps, such as venturi, diaphragm, or rotary-vane pumps.

An irrigation line 151 can be configured to discharge an irrigation liquid to the surgery site to equalize the pressure at the surgery site due to the aspirated liquid and cataract fragments. The irrigation tube can be coupled to an irrigation system 158 to supply the irrigation liquid. The components of the phaco handpiece can be disposed in a rigid housing 150. A controller 160 can be used to supply power and controlled signals to the phaco handpiece, e.g., to the driver system 131 to turn on or off or to adjust a power or frequency to the ultrasonic transducer, to the aspiration pumping system 141 to create an aspiration suction at the cannula, and to the irrigation system to supply the irrigation liquid to the surgery site. For example, the controller can include a power unit which controls the power supplied to the ultrasonic drive, including a frequency and a pulse-duty ratio of the ultrasonic signals. The power unit can receive control signals from a control unit such as a footswitch having a pedal or a switch embedded in the housing.

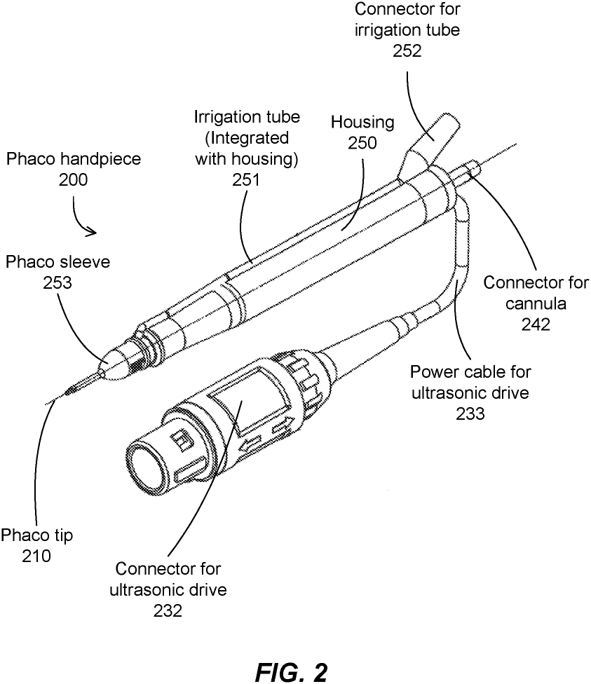

FIG. 2 illustrates a perspective view of a phaco handpiece according to some embodiments. A phaco handpiece 200 can be a medical handpiece for phacoemulsification in a cataract operation in ophthalmology. The phaco handpiece can be connected to a separate control (not shown) for controlling an aspiration pumping system (not shown), an irrigation system (not shown), and a power source (not shown). A power source can be removably connected to the phaco handpiece via a cable 233 through the connector 232. The irrigation system can be connected to the phaco handpiece through an irrigation connector 252, e.g., an irrigation interface of the irrigation tube 251 with the irrigation system. The aspiration pumping system can be connected to the phaco handpiece through an aspiration connector 242, e.g., a connector coupled to the cannula to connect the cannula to the aspiration pumping system.

The phaco handpiece 200 can be provided with a housing 250 coupled to a phaco sleeve 253, both of which can be configured to cover or surround the internal components of the phaco handpiece. The housing can include a polymer material, such as a plastic, and can be configured to support the internal components.

The internal components can include an ultrasonic drive, which can include an ultrasonic transducer configured to generate ultrasonic vibrations, for example, through piezo ceramic elements. The ultrasonic transducer can be coupled to a sonotrode for amplifying the ultrasonic vibrations. A phaco tip 210 can be coupled to the sonotrode to receive the amplified ultrasonic vibrations and to use the ultrasonic vibrations to emulsify materials at the surgery site, such as to break the cataracts into cataract fragments. A power cable 233 can be coupled to the ultrasonic drive, such as to the ultrasonic transducer. The coupling can be hardwired or can be connected through a connector to the ultrasonic transducer. At the other end of the cable 233, there can be a connector 232 to be connected to an ultrasonic driver system, to provide power and electrical control signals to the ultrasonic transducer.

Protruded from the phaco sleeve is a phaco tip 210 having an aspiration passage for removing cataract fragments and liquid at the surgery site through the tip of the phaco tip. The phaco tip can be introduced into the eye through a small incision. The aspiration passage in the phaco tip can be coupled to a cannula passing through the ultrasonic drive and protruded at the back end of the phaco handpiece. A connector 242 can be coupled to the protruded end of the cannula to link the aspiration passage from the tip of the phaco tip to an aspiration pumping system, to create an aspirating suction to remove the cataract fragments and liquid at the surgery site.

The phaco tip can be surrounded by a phaco sleeve 253. The phaco sleeve can include an irrigation port fluidly coupled an irrigation tube 251 to supply an irrigation liquid to the surgery site. For example, the irrigation port can be one or more holes are placed at the sides of the phaco sleeve towards the end of the sleeve so the irrigation liquid can be delivered into the surgery site close but in a direction away from the tip of the phaco tip.

The irrigation tube 251 can be integrated with the housing, and can be configured to deliver an irrigation liquid to the phaco sleeve to be delivered to the surgery site, The irrigation tube can include a connector 252 for connecting to an irrigation system, which can supply the irrigation liquid to the irrigation tube to be delivered to the surgery site at the tip portion of the phaco sleeve. The irrigation system can create and control a constant liquid flow to supply a predetermined amount of liquid to the phaco handpiece for use during surgical operation.

During the surgical procedure, the phaco tip can transfer ultrasonic energy into the patient’s eye, for example, to emulsify or break apart the cataract within the patient’s eye. Concurrently with the emulsification, irrigation liquid from the irrigation system can be delivered to the eye via the irrigation tube and the irrigation port on the phaco sleeve. Also during and after the emulsification, the irrigation liquid and emulsified cataract fragments can be aspirated from the eye by the aspiration pumping system via the aspiration passage in the phaco tip and the cannula.

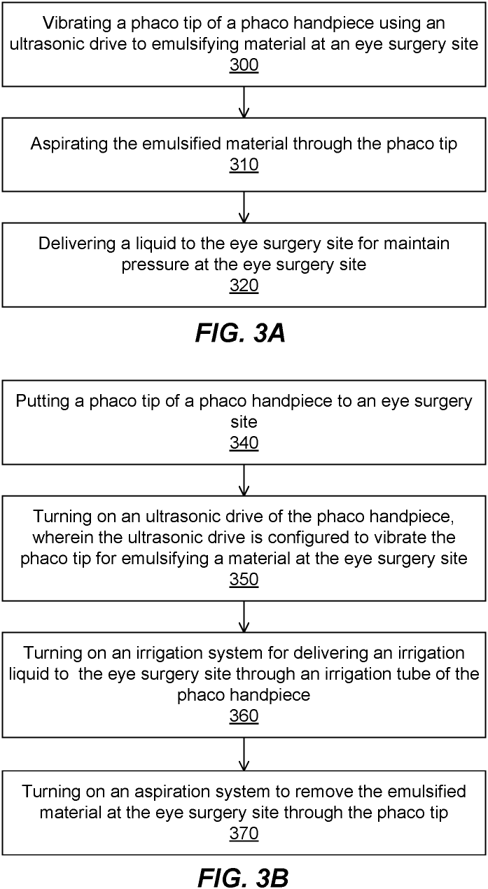

FIGS. 3A - 3B illustrates flow charts for operations of a phaco handpiece according to some embodiments. In a surgical treatment for cataract using an ultrasound phacoemulsification process, a phaco tip is inserted into the eye through an incision in the cornea. The cataract can be comminuted using the ultrasound-excited phaco tip, and the fragmented cataract is removed by aspiration, with the intraocular pressure at the eye being maintained by supplying an irrigation fluid.

A phaco handpiece can be configured to perform three tasks simultaneously. The tip of the phaco tip vibrates at an ultrasonic frequency to break apart and emulsify the cataract. An irrigation liquid, such as a saline solution, is fed into the eye to irrigate and to keep the eye from collapsing. And an aspiration line removes the liquid and emulsified pieces of the cataract.

In FIG. 3A, operation 300 vibrates a phaco tip of a phaco handpiece using an ultrasonic drive to emulsifying material at an eye surgery site. Operation 310 aspirates the emulsified material through the phaco tip. Operation 320 delivers a liquid to the eye surgery site for maintain pressure at the eye surgery site.

In FIG. 3B, operation 340 puts a phaco tip of a phaco handpiece to an eye surgery site. Operation 350 turns on an ultrasonic drive of the phaco handpiece, with the ultrasonic drive configured to vibrate the phaco tip for emulsifying a material at the eye surgery site. Operation 360 turns on an irrigation system for delivering an irrigation liquid to the eye surgery site through an irrigation tube of the phaco handpiece. Operation 370 turns on an aspiration pumping system to remove the emulsified material at the eye surgery site through the phaco tip.

### Assembling of a Phaco Handpiece

Non-disposable phaco handpieces can require rigorous cleaning after each surgery procedure due to contamination, for example, in the aspiration and irrigation pathways. The present disposable phaco handpiece can include disposable components, to allow for the disposal of components exposed or associated with the fluid pathways.

The disposable phaco handpiece can be configured to protect components from contamination with a design to allow components to be easily detached to be disposed or reused without any difficulties and with little effort.

In some embodiments, the phaco handpiece can include some disposable components, some reusable components, and some components that can be selected to be disposable or reusable. The reusable components in the phaco handpiece can be designed with adequate shielding to avoid contact with the contaminated materials, thus allowing the shielded components to be reusable. For example, since the ultrasonic drive can be an expensive component of the phaco handpiece, the ultrasonic drive can be designed with fluid-tight seals to eliminate contact with the aspiration an irrigation fluid and can be designed with good shielding to eliminate contact with the surgical ambient.

The disposable components in the phaco handpiece can be designed with low cost materials, such as the cannula, the irrigation tube, and the housing. Further, the phaco handpiece can be designed so that some components can be either reusable or disposable, depending on particular situations. For example, the phaco tip can be expensive, but it would be difficult to shield or isolate from the contaminated materials in the surgery site. As such, the phaco tip can be disposable in critical situations, or can be cleaned to be reused in situations not demanding critical cleanliness. Similarly, the power cable and the connector for the cannula can be disposed or reusable.

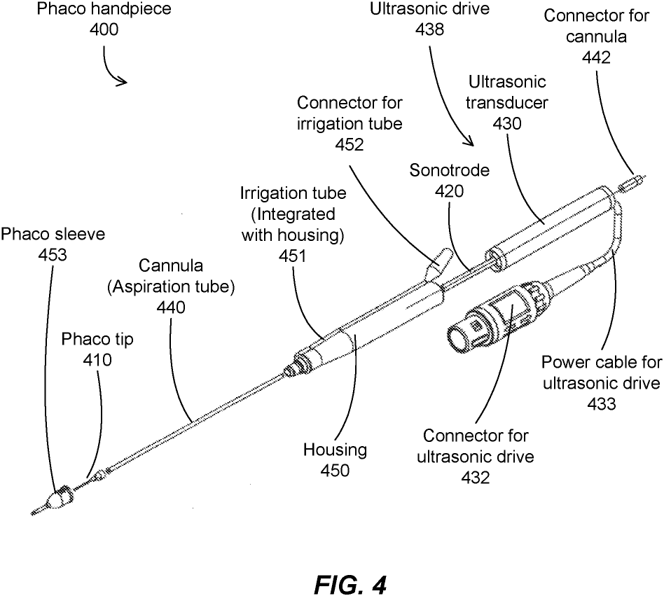

FIG. 4 illustrates an exploded view of a phaco handpiece according to some embodiments. The phaco handpiece 400 can include a vibration subassembly configured to generate ultrasonic vibrations at a phaco tip to emulsify or fragment cataract, an aspiration subassembly configured to aspirate the emulsified or fragmented cataract through an aspiration passage in the phaco tip, and an irrigation subassembly configured to deliver an irrigation liquid to the eye during the cataract surgery.

The vibration subassembly can include an ultrasonic drive 438 that includes an ultrasonic transducer 430 and a sonotrode 420. A phaco tip 410 having an aspiration passage can be coupled to the sonotrode 420 to receive the vibrations. The ultrasonic transducer can include a piezo crystal power unit to generate ultrasonic vibrations, which can be amplified by the sonotrode. The ultrasonic vibrations are then transferred to the cataract material via the phaco tip for fragmenting and emulsifying. The emulsified and fragmented cataract then can be aspirated and removes by the aspiration subassembly, such as through an aspiration passage in the phaco tip for discharging outside the patient body.

The ultrasonic drive can be the most expensive component of the phaco handpiece, and thus can be designed with shielding to be protected from the contaminated fluid and also from the surgical ambient. The ultrasonic unit thus can be reconditioned, e.g., removed from the used phaco handpiece to be cleaned and sterilized before being assembled in a new phaco handpiece. For example, a disposable cannula 440 can be disposed along a central axis of the ultrasonic drive, which can shield the inside of the ultrasonic drive from the emulsified and fragmented cataract aspired from the ophthalmic surgery site. In addition, the phaco tip and the cannula can be designed to have a leak-free coupling to prevent the contaminated waste in the fluid pathway to contact the ultrasonic drive to contaminate the ultrasonic drive. Further, the ultrasonic drive can be disposed in a disposable housing 450, which can shield the outside of the ultrasonic drive from the surgical environment and ambient. The disposable housing can be designed with an integrated irrigation tube, which can shield the ultrasonic drive from the irrigation liquid delivered to the ophthalmic surgery site.

A cable 433 can be integrated or coupled to the ultrasonic drive 438, e.g., to the ultrasonic transducer 430, to provide power, including control signals, to the piezo crystal from a driver system. One end of the cable can be hardwired to the ultrasonic transducer, or can be removably coupled to the ultrasonic transducer using a transducer connector (not shown). The opposite end of the cable can include another connector 432, which can be used to removably couple to the driver system.

The cable is not expensive and in general, not exposed to the contaminated fluid. The cable can be exposed to the surgical environment and ambient, which is not as serious as being exposed to the contaminated fluid. Thus, the cable can be configured to be disposable, e.g., to be single use and to be discarded after every surgical procedure. Alternatively, the cable can be shielded from the contaminated fluid and from the surgical environment, to allow the cable to be reused, e.g., removed from the used phaco handpiece to be assembled in a new phaco handpiece, after an optional cleaning step. For example, the transducer connector and a portion of the cable can be shielded inside the housing that shields the ultrasonic drive. The remaining portion of the cable can also be shielded, such as being plastic wrapped, to allow the cable to be reused without or with minimum cleaning, for example, by replacing the plastic wrap.

The aspiration subassembly can include the phaco tip 410 of the vibration subassembly, a cannula 440, e.g., an aspiration tube, coupled to the phaco tip. A connector 442 can also be coupled to the cannula to provide connectivity to an aspiration pumping system to provide the aspiration power. The phaco tip can have an aspiration passage, which is fluidly communicated with the aspiration conduit in the cannula. After the cataract is emulsified and fragmented by the vibrations of the phaco tip, the emulsified and fragmented cataract is then aspirated and removes through the aspiration passage in the phaco tip to the aspiration conduit in the cannula to be discharged to the aspiration pumping system.

The ultrasonic drive can be hollow throughout to receive the cannula, which is configured to provide a passage for the emulsified fluid to be discharged to the aspiration pumping system after entering the aspiration passage in the phaco tip. The cannula can be configured to be inserted into the ultrasonic drive along the axis of the ultrasonic drive, e.g., the cannula can also be removed from the ultrasonic drive to be disposed after a surgical procedure.

The cannula can be configured to be single use, e.g., the cannula can be disposed after being used in an eye surgery since the contaminated waste is conducted through the inside of the cannula. For example, the cannula can be designed to make a tight seal with the ultrasonic drive, both at the coupling to the sonotrode and also at the connection to the aspiration pumping system. The cannula can be made from soft materials, which can have the advantage of not causing wear or damage to the sonotrode when forming the seal with the sonotrode, and which can allow the sonotrode to be reused for multiple times. For example, cannula can be made from a softer material than the material of the sonotrode, to prevent wear and damage to the sonotrode when replacing the seal. In some embodiments, the cannula can be made from a hard material, such as harder than the sonotrode or as hard as the sonotrode.

A share component between the ultrasonic subassembly and the aspiration subassembly is the phaco tip. The phaco tip can be coupled to the cannula of the aspiration subassembly and also to the sonotrode of the ultrasonic subassembly. The phaco tip can be constructed in the form of a hollow needle with a small needle tip for ease of entering the small incision at the eye. The phaco tip is configured to be coupled to the cannula so that the hollow portion of the phaco tip connects with the cannula to form a fluid pathway for removing the emulsified fluid at the surgery site.

The phaco tip can be configured to break the cataract into pieces and then emulsify and aspirate the emulsified or cataract pieces to the aspiration pumping system. To break the cataract, the phaco tip can be coupled to the ultrasonic drive, which includes an ultrasonic transducer to convert electrical energy applied to the ultrasonic transducer into mechanical movements of the phaco tip. The ultrasonic transducer can include piezo crystals, which can oscillate in response to an applied high frequency current. The oscillation can be transmitted to the phaco tip via a sonotrode, which functions as an amplifier, to provide vibration movements of the phaco tip. The movements of the phaco tip can reach a maximum of 100 to 120 µm, with a working frequency ranging from 26 to 45 kHz.

The phaco tip can be expensive and is exposed to the contaminated fluid, e.g., the phaco tip functions to aspirate the emulsified and fragmented cataract to be disposed away from the eye. Thus, since the phaco tip is in direct contact with the contaminated waste, the phaco tip can be disposable, e.g., to be single use and to be discarded after every surgical procedure to prevent cross contamination. Alternatively, since the phaco tip can be an expensive component of the phaco handpiece, the phaco tip can be cleaned and sterilized after each use, which can allow the phaco tip to be re-assembled with other new or clean components of the phaco handpiece to form a new phaco handpiece.

The irrigation subassembly can include an irrigation tube 451 integrated in a housing in which the ultrasonic drive is placed. One end of the integrated irrigation tube is shaped to form a connector 452, such as a Luer female type connector, to provide connectivity to an irrigation system. The housing is also shaped to have an opening at an opposite end of the irrigation tube, in which at least a portion of the phaco tip is protruded. A phaco sleeve 453 can be coupled to the opening to cover most of the phaco tip, with just a small portion of the tip of the phaco tip exposed to perform emulsification. The phaco sleeve can be configured to be coupled with the irrigation tube, to allow the irrigation liquid to pass to the phaco sleeve and into the eye during the cataract surgery.

The housing and the phaco sleeve can be configured to be disposable, e.g., single use since the irrigation liquid is conducted through the irrigation tube, which is integrated with the housing. The irrigation liquid can also run inside the phaco sleeve to deliver to the eye at exit ports in the vicinity of the tip of the phaco tip. For example, the housing and the phaco sleeve can be designed to make a tight seal with each other. Further, the housing can be designed to make a seal with the ultrasonic drive at the phaco tip to prevent contamination, due to the irrigation liquid in the phaco sleeve. The phaco sleeve can be designed to make a tight fit with the tip of the phaco tip, to force the irrigation liquid to exit the phaco sleeve through the exit ports.

The housing and the phaco sleeve can be configured to shield most of the ultrasonic drive from being exposed to the surgical ambient. For example, the housing and the phaco sleeve can be configured to cover the ultrasonic drive and the phaco tip, leaving only a tip portion of the phaco tip for performing emulsification of the cataract and for aspirating the emulsified cataract.

In some embodiments, the housing can be configured to expose an end face of the ultrasonic drive to the ambient. Alternatively, a housing end can be configured to cover the ultrasonic drive end face, together with a portion of the aspiration connector 442 and the power cable 433.

The irrigation-integrated housing and the phaco sleeve can be made from inexpensive materials, such as plastic, to allow the use of disposable irrigation tube in the phaco handpiece. An advantage of a plastic housing is the low transfer of vibration energy, thus reducing potential vibration loss generated from the ultrasonic drive. Further, ergonomic improvements for the housing can be rapidly designed with plastic housings.

Before the phaco handpiece can be used, the components of the phaco handpiece can be assembled together. The assembled components can be new or reconditioned components, which can be kept in sterile packaging to ensure the sterility of the phaco handpiece to be used on a new patient in a simple and safe manner.

After the surgical procedure, the components can be dismantled, with the disposable components disposed, the reusable components cleaned and sterilized, and the disposed or reusable components selected to be disposed or to be cleaned and sterilized. The phaco handpiece can be designed to be quickly and easily assembled and disassembled.

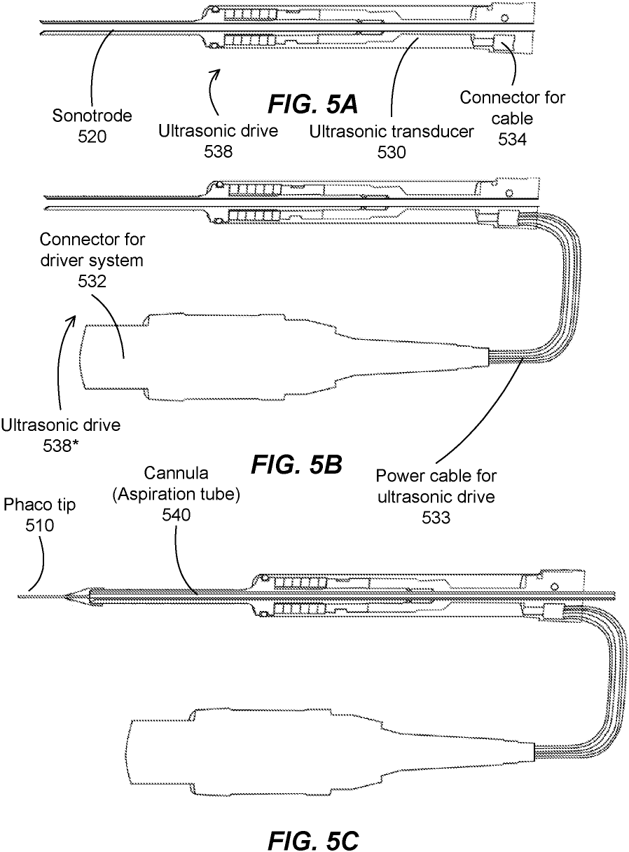

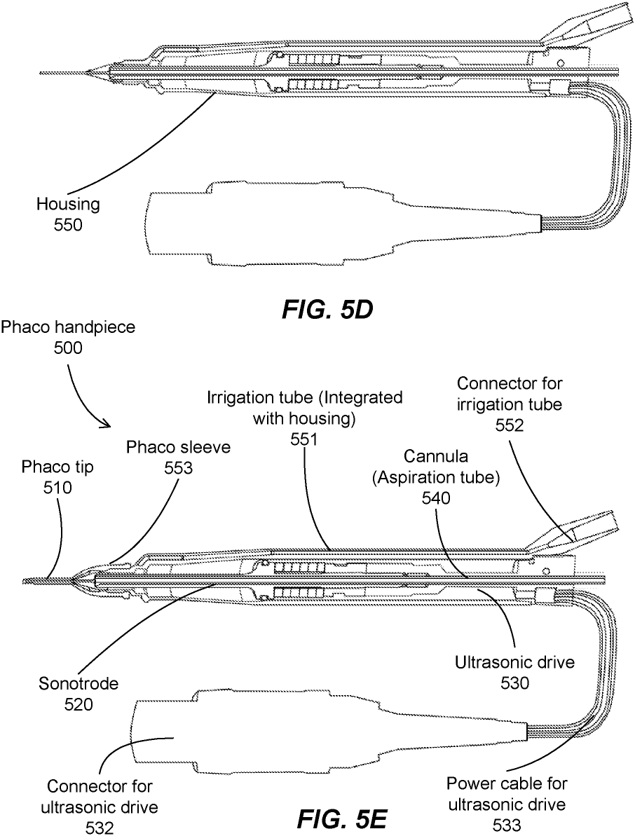

FIGS. 5A - 5E illustrate an assembling process for a phaco handpiece according to some embodiments. FIG. 5A shows an ultrasonic drive 538 having an ultrasonic transducer 530 coupled to a sonotrode 520. The sonotrode can be integrated to the ultrasonic transducer, e.g., the ultrasonic transducer and the sonotrode is configured to be a single unit for an efficient transfer of ultrasonic energy from the ultrasonic transducer to the sonotrode. The ultrasonic drive 538 can include a connector 534 for connecting to an separate cable. The ultrasonic drive can be new or reconditioned.

FIG. 5B shows an alternate ultrasonic drive 538* with an integrated cable 533 having a connector 532 to be connected to a driver system. In the previous figure, the ultrasonic drive is configured to be without an integrated cable, meaning a separate new or reusable cable 533 with the connector 532 at one end and another connector at the opposite end configured to be coupled to the connector 534.

FIG. 5C shows an ultrasonic drive assembled with a cannula 540 and a phaco tip 510. A cannula 540 can be releasably pushed into the ultrasonic drive, such as pushing into a central channel in the ultrasonic drive. The releasable cannula can be configured to be a disposable part. The cannula can be completely inserted so that one end of the cannula is in a sealing contact with the sonotrode.

A new or reused, e.g., cleaned and sterilized, phaco tip can be coupled to the sonotrode and the cannula. The phaco tip can be hollow, e.g., having an aspiration passage in the phaco tip configured to aspirate the cataract fragments. The aspiration passage of the phaco tip can be configured to make a tight fluid communication with the cannula 540, for example, by pressing the phaco tip against the aspiration tip. The phaco tip can also be coupled to the sonotrode, such as forming a tight seal to prevent any liquid at the tip of the phaco tip to contaminate the ultrasonic drive, such as the emulsified or fragmented cataract or the irrigation liquid supplied to the tip of the phaco tip.

For example, the phaco tip can have inner thread, which can be screwed to the outer threaded end of the sonotrode. In addition, the force caused by the threading action can be used to press the phaco tip against the cannula, e.g., to form the tight seal for the fluid communication between the aspiration passage of the phaco tip and the aspiration passage in the cannula.

Other assembling process can be used, such as the phaco tip making a snap coupling, a magnetic coupling, or a press fit coupling with the sonotrode. The coupling between the phaco tip to the cannula, and the coupling between the phaco tip and the sonotrode can provide tight seals to prevent contamination of the ultrasonic drive.

FIG. 5D shows a housing 550 assembled with the ultrasonic drive, the cannula and the phaco tip. The assembly of the ultrasonic drive, the cannula and the phaco tip can be releasably inserted into a housing 550, for example, from a larger end of the housing. At the opposite smaller end of the housing, the housing can be configured to make seal with a base portion of the phaco tip.

The housing can have a limiter in the inner surface, which serves as a stop for the insertion of the ultrasonic drive. The housing can also have support elements for holding the ultrasonic drive in fixed positions in the housing. The main support elements can be positioned on the ultrasonic transducer, such as through vibration damping material configured to not disturb the vibration transfer from the ultrasonic transducer to the sonotrode and to the phaco tip. Support elements in the form of flexible fins can be used to couple the housing to the sonotrode, to allow the sonotrode to vibrate.

FIG. 5E shows a phaco sleeve 553 assembled to the housing 550 at the smaller end of the housing. For example, the phaco sleeve can slide along the phaco tip to couple with the end of the housing. The phaco sleeve can have a fluid interface, which is configured to be coupled with the irrigation tube to receive the irrigation liquid. The phaco sleeve can have one or more exit ports near the tip of the phaco tip to allow the irrigation liquid to exit from the phaco sleeve.

With the housing and the phaco sleeve designed to shield the internal components, the ultrasonic drive does not come into contact either with the patient or with the aspiration and irrigation liquids and can therefore be reused without or with minimum need to be cleaned or sterilized. The cable can also be shielded to be reused without or with minimum cleaning or sterilizing. Alternatively, the separate cable can be disposed after each use for critical cleanliness requirements since the cable is not an expensive item. The phaco tip is always exposed to the cataract, and therefore can be disposed or reused after being cleaned and sterilized. The housing with the integrated irrigation tube, the phaco sleeve, and the cannula can be single use components. With the present design, the sterility of the phaco handle is achieved without costly replacements or sterilization process.

After finish assembling, the phaco handpiece can be coupled to the driver system through the cable connector 534, to the aspiration pumping system with a cannula connector, and to the irrigation system through the irrigation connector 552.

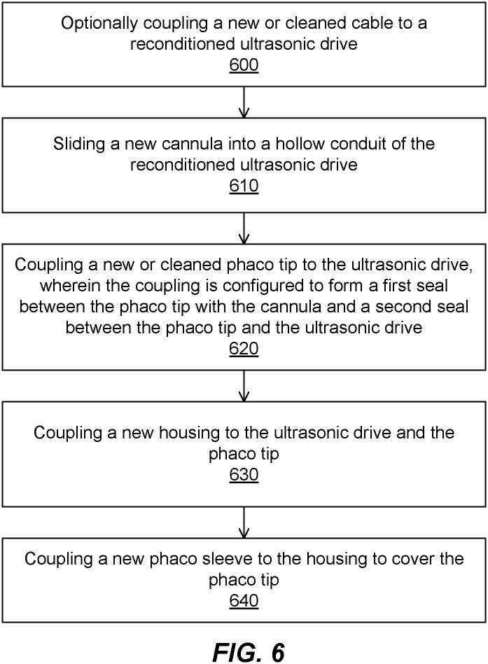

FIG. 6 illustrates an assembling process for a phaco handpiece according to some embodiments. Operation 600 optionally couples a new or cleaned cable to a reconditioned ultrasonic drive. Operation 610 slides a new cannula in a hollow conduit of the reconditioned ultrasonic drive. Operation 620 couples a new or cleaned phaco tip to the ultrasonic drive, with the coupling configured to form a first seal between the phaco tip with the cannula and a second seal between the phaco tip and the ultrasonic drive. Operation 630 couples a new housing to the ultrasonic drive and the phaco tip. Operation 640 couples a new phaco sleeve to the housing to cover the phaco tip.

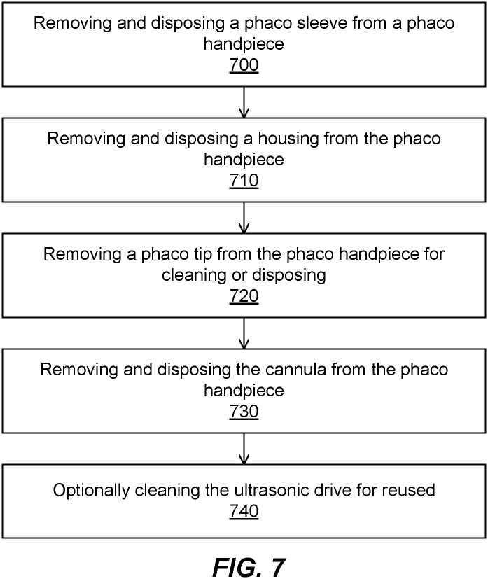

FIG. 7 illustrates a disassembling process for a phaco handpiece according to some embodiments. Operation 700 removes and disposes a phaco sleeve from a phaco handpiece. Operation 710 removes and disposes a housing from the phaco handpiece. Operation 720 removes a phaco tip from the phaco handpiece for cleaning or disposing. Operation 730 removes and disposes the cannula from the phaco handpiece. Operation 740 optionally cleans the ultrasonic drive for reused.

### A Cannula in an Ultrasonic Drive and Their Interface

In some embodiments, the present invention discloses a phaco handpiece having a cannula removably coupled along a central axis of an ultrasonic drive in a configuration that can allow the cannula to function as a barrier between the aspiration fluid and the ultrasonic drive, which can prevent the ultrasonic drive from being contaminated with the aspiration fluid, and thus can be reused, without or with minimum cleaning.

In addition for preventing contamination, the cannula can be configured so that the ultrasonic vibration generated by the ultrasonic drive is efficiently transfer to a phaco tip, e.g., without or with minimum energy loss due to the interface between the cannula and the ultrasonic drive.

In some embodiments, the interface between the ultrasonic drive and the cannula can be configured to not disturb the vibration transfer, e.g., from the ultrasonic transducer to the sonotrode to the phaco tip, with or without through the cannula. For example, the friction between the two surfaces, e.g., the outer surface of the cannula and the inner surface of the sonotrode can be configured to be minimized to prevent the loss of vibration energy when transferring from the sonotrode to the phaco tip through the cannula.

The efficient transfer of energy to the phaco tip can reduce heat generation at the phaco tip, which can beneficial to the patients. Furthermore, the phaco handpiece can be designed with low acoustic impedance materials, which can improve the effective coupling of ultrasonic energy to reduce energy transfer loss in the form of heat generation.

The phaco handpiece can include a hollow ultrasonic drive, with the hollow portion configured to receive a cannula, e.g., a tube for aspirating the aspirated fluid. The cannula can be configured to be removably inserted into the ultrasonic drive along an axis of the ultrasonic drive, e.g., the cannula can also be removed from the ultrasonic drive for replacement. The cannula can have a sealable coupling at one end, such as at the end coupled to the sonotrode. The sealable coupling can be configured to be coupled with the tip end of the sonotrode, to prevent the cannula from slipping away from the ultrasonic drive and to prevent the aspirated fluid from contacting the hollow portion of the ultrasonic drive.

The configuration of the cannula inserted in the ultrasonic drive can be configured to achieve an effective vibration transfer, for example, accomplished by a small gap between the cannula and the sonotrode, together with an optimum friction between the cannula and the sonotrode. The friction can be reduced by the cannula or the sonotrode having a low friction surface structure, such as made from a low friction material.

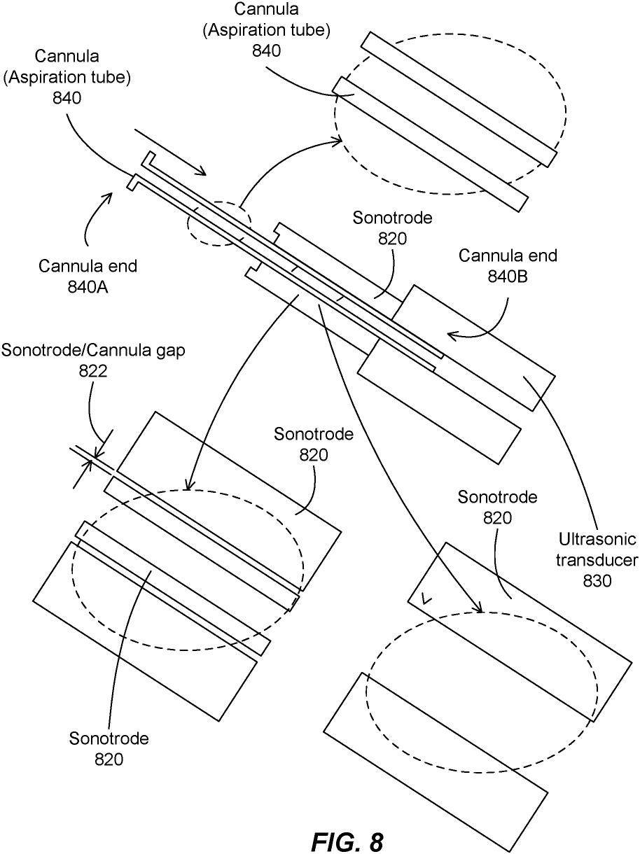

FIG. 8 illustrates an effective energy transfer with a small gap between a cannula and an ultrasonic drive according to some embodiments. A cannula 840 can be inserted into the hollow portion of an ultrasonic drive, such as from the sonotrode 820 toward the ultrasonic transducer 830. The cannula 840 can be inserted into the ultrasonic drive using a first end 840B and inserted from the sonotrode toward the transducer. Opposite of this end is a second end 840A, which can be configured to be coupled to the tip or end of the sonotrode.

The outer diameter of the cannula can be just slightly smaller than the inner diameter of the hollow portion of the sonotrode, e.g., there is a gap 822 between the cannula and the sonotrode when the cannula is assembled in the ultrasonic drive. The gap can be large enough for ease of assembling the cannula to the ultrasonic drive, and can be small enough to allow an effective energy transfer from the sonotrode to the cannula. For example, the gap can be less than 0.5 mm, less than 0.2 mm, less than 0.1 mm, less than 0.05 mm, less than 0.02 mm, or less than 0.01 mm. The gap can be designed as a tradeoff between ease of assembly/disassembly and effective energy transfer.

In addition to the small gap between the cannula and the sonotrode, the surface roughness of the cannula and the sonotrode can also be controlled or optimized to achieve an effective energy transfer, e.g., with minimum loss of vibration transfer from the sonotrode to the cannula.

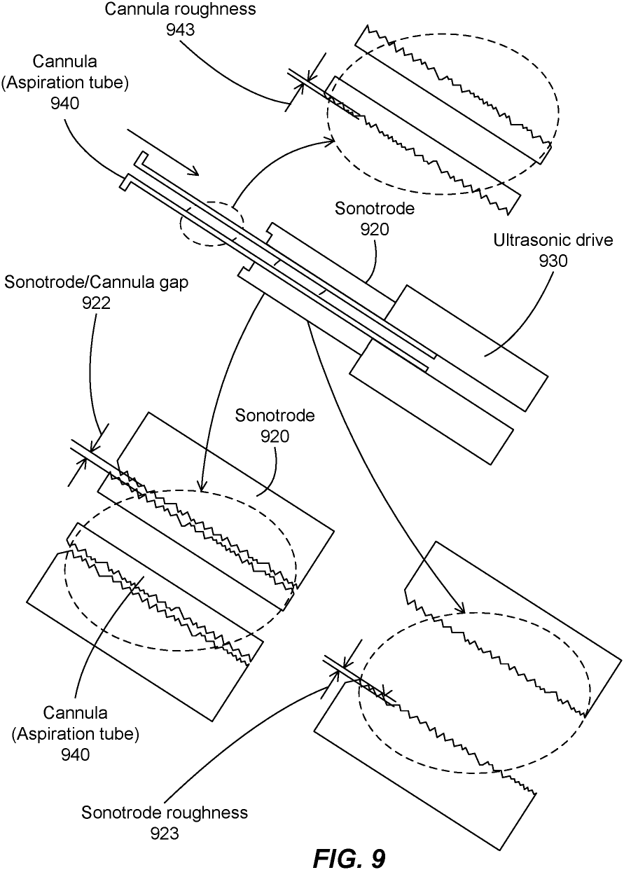

FIG. 9 illustrates an effective energy transfer with a small gap between a cannula and an ultrasonic drive according to some embodiments. A cannula 940 can be inserted into the hollow portion of an ultrasonic drive, such as from the sonotrode 920 toward the ultrasonic transducer 930.

The surface roughness 943 of the outer surface of the cannula and the surface roughness 923 of the inner surface of the hollow portion of the sonotrode can be controlled to prevent excessive loss of vibration transfer from the sonotrode to the cannula, e.g., to achieve an effective energy transfer to lower the heating of the components of the ultrasonic drive. For example, the surface roughness of the cannula and the sonotrode can be less than Ra 50 microns, or less than Ra 20 or 10 microns, with Ra being the average surface roughness. In addition, the gap, accounted for the surface roughness, between the cannula and the sonotrode can also be small to prevent loss of ultrasonic energy transfer, for example, the gap can be less than 100 microns, less than 50 microns, or less than 20 microns.

In some embodiments, the phaco handpiece can include a sonotrode coupled with a cannula made from a softer material, an equally hard material, or a harder material. In some embodiments, the coupling between the cannula and the sonotrode can include a tight seal, e.g., a seal to prevent a leakage of fluid, such as the aspiration fluid running in the cannula. A seal of any type can be used, including a direct form fit without any additional sealing material.

A flange seal at the end of the cannula can be coupled with a mated recess on the sonotrode. The flange seal between the cannula and the sonotrode can be formed using a force or pressure applied to the seal, for example, by a phaco tip pushing on the cannula when coupling with the sonotrode. The seal is configured to prevent contaminated waste in the cannula from leaking to the ultrasonic drive.

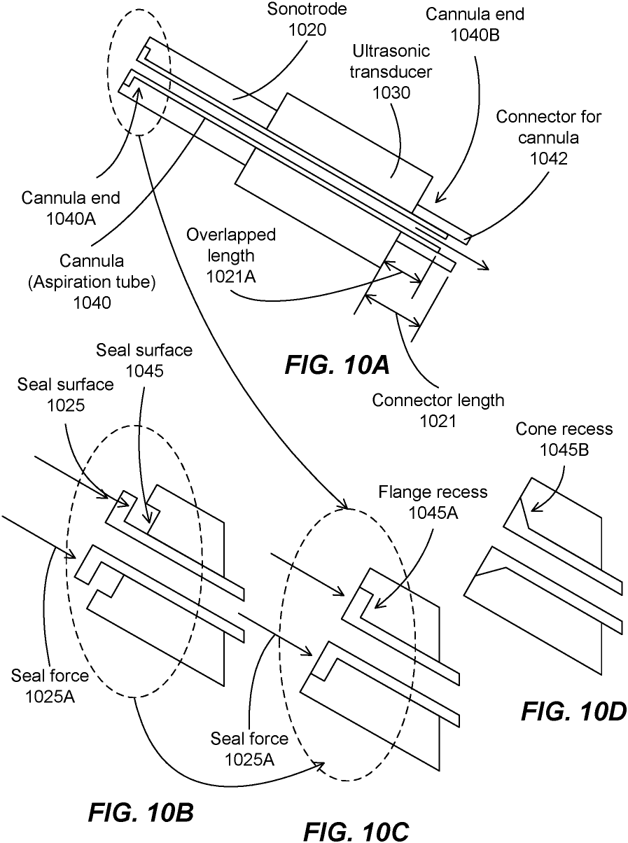

FIGS. 10A - 10D illustrate seal configurations between a cannula and a sonotrode according to some embodiments. A cannula 1040 can be inserted into the hollow portion of an ultrasonic drive, such as from the sonotrode 1020 toward the ultrasonic transducer 1030. The cannula 1040 can be inserted into the ultrasonic drive using the first end 1040B and inserted from the sonotrode toward the transducer. Opposite of the first end is the second end 1040A, which can be configured to be coupled to the tip or end of the sonotrode. The cannula can be coupled to the sonotrode at the second end 1040A, and can be protruded from the ultrasonic transducer at the first end 1040B to be coupled to an aspiration pumping system using a connector 1042.

A direct form fit between the cannula and the sonotrode can be configured between the second end 1040A of the cannula and a tip end of the sonotrode. The second end 1040A of the cannula can include a sealing surface 1025, which is configured to be mated with a corresponding sealing surface 1045 at the tip end of the sonotrode. For example, the sealing surface 1025 can have a flange shape or a cone shape, to be mated with a recess shape 1045A or a cone shape 1045B of the sonotrode tip end. Other shapes can be used.

For connecting the cannula with an aspiration pumping system, the cannula can be protruded from the ultrasonic transducer, and a connector 1042 can be coupled to the protruded end, e.g., the first end 1042B of the cannula, to couple the cannula with an aspiration pumping system. The connector 1042 can make a seal with the cannula, such as having a Luer connection to be attached leak-free to the cannula.

In some embodiments, the length 1021 of the connector 1042, or specifically, the overlapped length 1021A of the connector 1042 with the cannula, The length of the connecting piece can be optimized to provide a leak-free seal with the cannula during the surgical operation, e.g., to remain a leak-free seal even with the various movements of the phaco handpiece. The connector length 1021 or the overlapped length 1021A can be depended on the materials of the cannula and the connector, e.g., on the friction between the cannula and the connector, and can be greater than 5 mm, greater than 7 mm, greater than 9 mm, greater than 10 mm, greater than 12 mm, or greater than 15 mm.

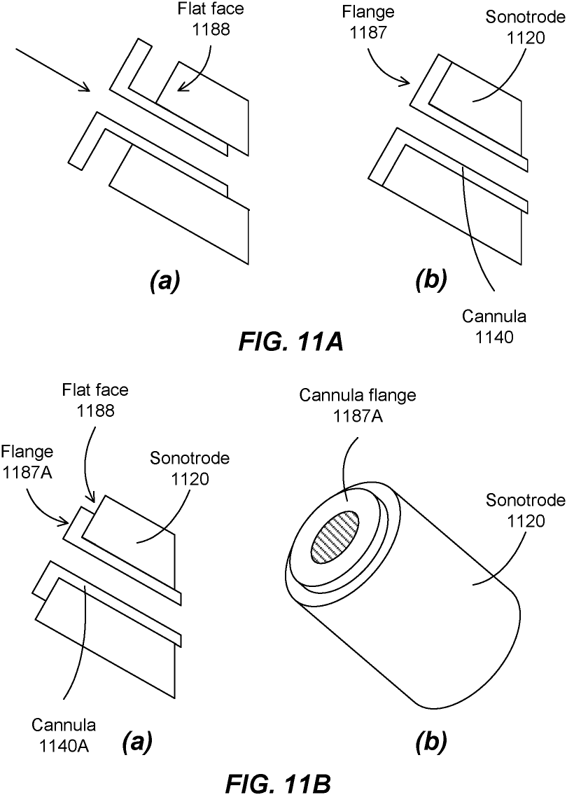

FIGS. 11A - 11B illustrate other seal configurations between a cannula and a sonotrode according to some embodiments. In FIG. 11A(a) and 11A(b), a cannula 1140 can have a flat flange 1187, which is configured to make a seal with a flat face 1188 of a sonotrode 1120. The flange 1187 can be as large as the flat face 1188 of the sonotrode. In some embodiments, the flange 1187 can be larger than the flat face 1188 of the sonotrode.

FIG. 11B(a) and 11B(b) show a cross section view and a perspective view of another seal between a cannula and a sonotrode. The cannula 1140A can have a flat flange 1187A, which is smaller than the flat face 1188 of the sonotrode 1120.

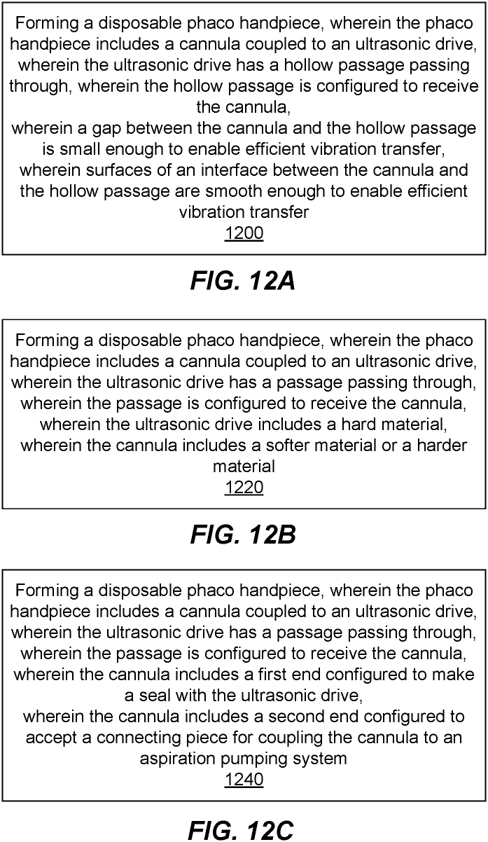

FIGS. 12A - 12C illustrate flow charts for coupling a cannula, e.g., an aspiration tube, with an ultrasonic drive according to some embodiments. In FIG. 12A, operation 1200 forms a phaco handpiece, with the phaco handpiece having a cannula coupled to an ultrasonic drive. The ultrasonic drive can have a hollow passage passing through, with the hollow passage configured to receive the cannula. A gap between the cannula and the hollow passage is configured to be small enough to enable efficient vibration transfer. Surfaces of an interface between the cannula and the hollow passage are configured to be smooth enough to enable efficient vibration transfer.

In FIG. 12B, operation 1220 forms a phaco handpiece, with the phaco handpiece having a cannula coupled to an ultrasonic drive. The ultrasonic drive can have a passage passing through, with the passage configured to receive the cannula. The ultrasonic drive can include a hard material. The cannula can include a softer material. In some embodiments, the cannula can include a harder material.

In FIG. 12C, operation 1240 forms a phaco handpiece, with the phaco handpiece having a cannula coupled to an ultrasonic drive. The ultrasonic drive can have a passage passing through, with the passage configured to receive the cannula. The cannula can include a first end configured to make a seal with the ultrasonic drive. The cannula can include a second end configured to accept a connecting piece for coupling the cannula to an aspiration pumping system.

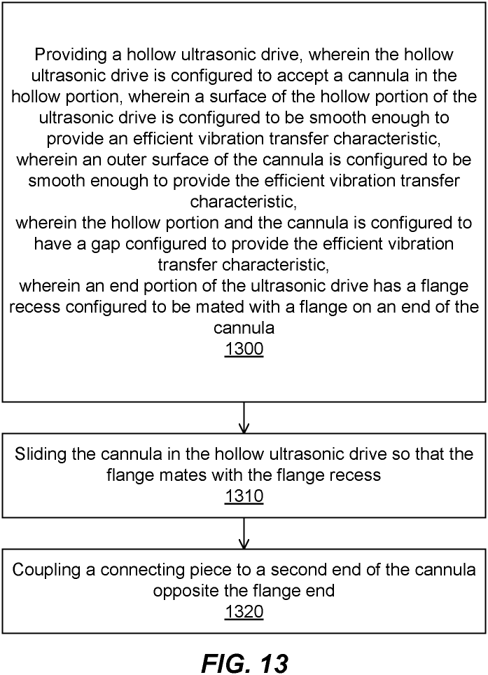

FIG. 13 illustrates a flow chart for forming a seal between a cannula, e.g., an aspiration tube, and an ultrasonic drive according to some embodiments. Operation 1300 provides

a hollow ultrasonic drive, with the hollow ultrasonic drive configured to accept a cannula in the hollow portion, with a surface of the hollow portion of the ultrasonic drive configured to be smooth enough to provide an efficient vibration transfer characteristic. An outer surface of the cannula is configured to be smooth enough to provide the efficient vibration transfer characteristic. The hollow portion and the cannula are configured to have a gap configured to provide the efficient vibration transfer characteristic. An end portion of the ultrasonic drive has a flange recess configured to be mated with a flange on an end of the cannula.

Operation 1310 slides the cannula in the hollow ultrasonic drive so that the flange mates with the flange recess. Operation 1320 couples a connecting piece to a second end of the cannula opposite the flange end.

### Interfaces of a Phaco Tip With a Cannula in a Phaco Handpiece

In some embodiments, the phaco handpiece can include a phaco tip configured to form a seal with both the sonotrode and the cannula disposed within the ultrasonic drive. The phaco tip can have an aspiration passage passing through the phaco tip. The aspiration passage is configured to make a seal with the cannula, to allow the fluid from the surgery site to be removed to an aspiration pumping system. The phaco tip can also include another seal with the sonotrode, which is configured to prevent contamination of the sonotrode and to improve the ultrasonic energy transfer.

The phaco tip thus can include a direct form fit seal with the cannula around the aspiration passage in the phaco tip. The direct form fit seal can be a seal with no sealing material, e.g., the phaco tip is pressed against the cannula to form the seal. Alternatively, a sealing element, such as a compressible material such as plastic or rubber, can be placed between the phaco tip and the cannula to form the seal.

The phaco tip can also include another seal with the sonotrode, such as a threaded seal, a snap seal, a press fit seal, or a magnetic seal. The seal with the sonotrode can be configured to provide a force acting on the form fit seal of the phaco tip with the cannula, which can ensure a pressure on the form fit seal to maintain the seal structure.

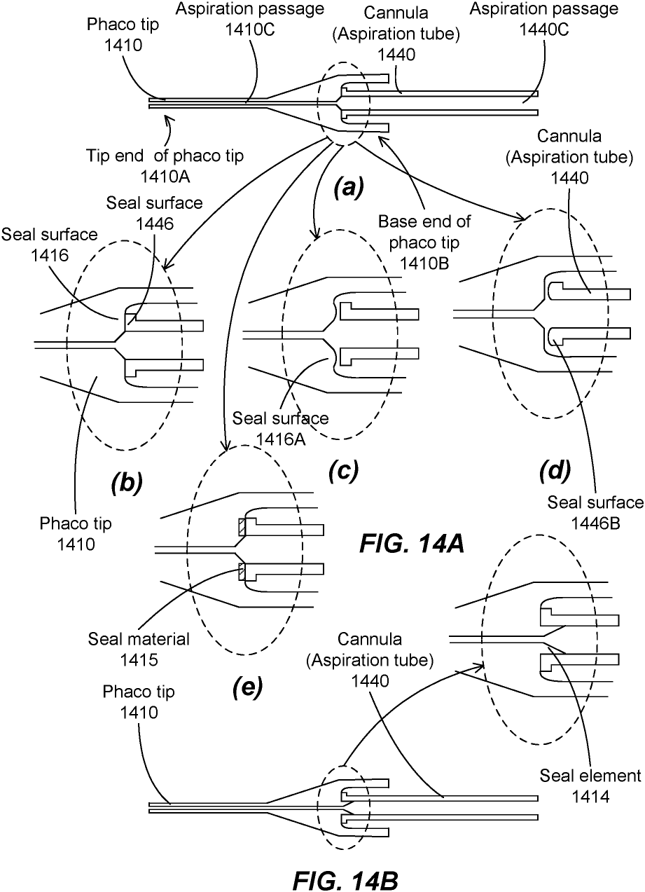

FIGS. 14A - 14B illustrate configurations for seal structures between a phaco tip and a cannula according to some embodiments. A phaco tip 1410 can include a needle tip 1410A, e.g., a tip end portion of the phaco tip, a base end portion 1410B configured to be coupled to the sonotrode, and an aspiration passage 1410C passing through the phaco tip. The phaco tip 1410 can have a seal surface 1416 surrounding the aspiration passage 1410C. The seal surface 1416 is configured to make a seal with a mating seal surface 1446 of the cannula 1440. The seal surface 1446 can be at one end of the cannula and can surround the aspiration passage 1440C of the cannula. The seal between the seal surfaces 1416 and 1446 is configured to make a leak-free fluid communication between the aspiration passage 1410C of the phaco tip and the aspiration passage 1440C of the cannula.

FIG. 14A(a) shows a direct form fit seal between the phaco tip and the cannula, e.g., the sealing surface 1416 of the phaco tip is pressed against the sealing surface 1446 of the cannula to form the seal. The pressing force can be generated by the phaco tip coupling with the sonotrode, which will be described in later section.

The seal surfaces 1416 and 1446 can include flat surfaces, which are configured to be pressed together to form a direct form fit seal (FIG. 14A(b)). The seal surface 1416A of the phaco tip can include a convex surface, configured to press on a flat surface of the seal surface of the cannula (FIG. 14A(c)). The seal surface of the phaco tip can include a flat surface, configured to press on a convex surface of the seal surface 1446B of the cannula (FIG. 14A(d)). The seal surface of the phaco tip can include a seal material 1415, which can be a compressible material for pressing on the seal surface of the cannula (FIG. 14A(e)).

FIG. 14B shows another configuration of the phaco tip, in which the seal surface can include a seal element 1414 protruded into the aspiration passage of the cannula, e.g., the seal surface of the phaco tip can be configured to wrap around the seal surface of the cannula. Other seal surfaces can be used to form a direct press fit seal between the phaco tip and the cannula.

In some embodiments, the phaco tip and the cannula can be mated together to form an integrated cannula. For example, the phaco tip and the cannula can be formed as a single unit, e.g., the phaco tip and the cannula are fabricated from a same material.

Alternatively, the phaco tip and the cannula can be two separate components that can be bonded together, such as at the sealing surface, for example, to form a secure coupling between the two components. In some embodiments, the phaco tip can be made of titanium, and the cannula made of steel or stainless steel. The phaco tip and the cannula then can be securely coupled together, for example, by a welding process including a friction welding process, a gluing process using an adhesive, by crimping together , by a press fit process.

After being used, the integrated cannula with the phaco tip can be discarded, or cleaned and sterilized to be reused. Alternatively, the cannula portion can be cut away from the integrated phaco tip/cannula, and the phaco tip can be reconditioned for securely coupled with a new cannula.

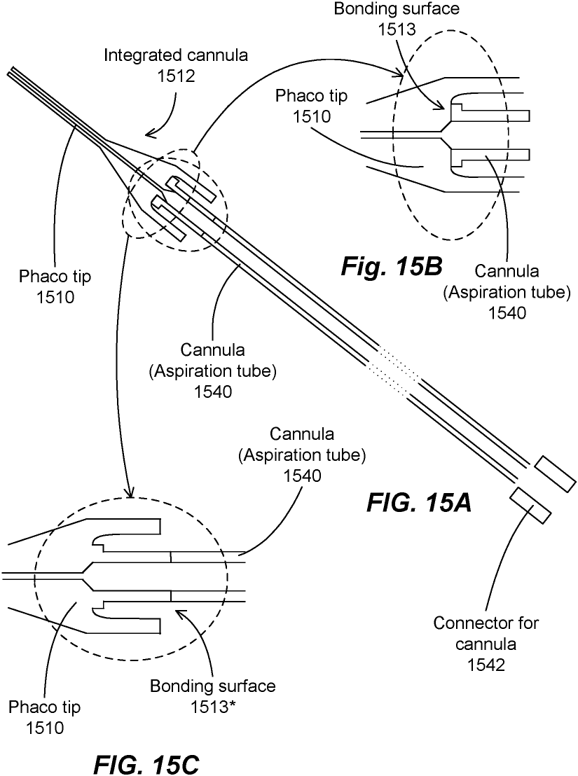

FIGS. 15A - 15C illustrate a configuration for an integrated phaco tip and cannula according to some embodiments. In FIG. 15A, an integrated cannula 1512 can include a phaco tip 1510 bonded to a cannula 1540, for example, by welding or by using an adhesive at a bonding surface 1513* and 1513 between the phaco tip and the cannula. The bonding can be permanent, e.g., for securely coupling the phaco tip with the cannula.

In assembling, the integrated cannula can be inserted into an ultrasonic drive from the end opposite to the phaco tip. The integrated cannula can protrude from the ultrasonic drive, which is configured for the cannula to be connected to an aspiration pumping system through a connector 1542.

FIG. 15B shows a coupling configuration between a phaco tip and a cannula. The bonding surface 1513 can be disposed between a surface surrounding the aspiration path of the phaco tip, and a surface surrounding the aspiration path of the cannula. An adhesion material can be used between the two bonding surfaces.

FIG. 15C shows another coupling configuration between a phaco tip and a cannula. The bonding surface 1513* can be disposed between and extended portion of the phaco tip, and the cannula. The extended tube of the phaco tip can be welded to the tube end of the cannula.

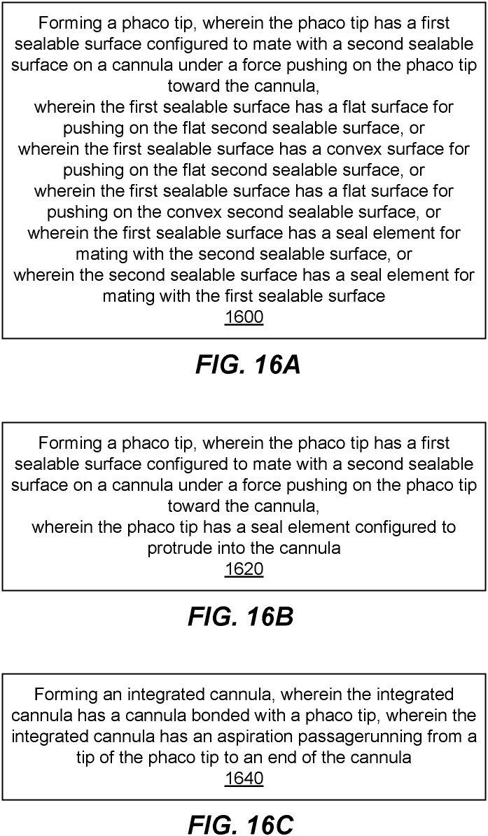

FIGS. 16A - 16C illustrate flow charts for forming a seal between a phaco tip and a cannula according to some embodiments. In FIG. 16A, operation 1600 forms a phaco tip, with the phaco tip having a first sealable surface configured to mate with a second sealable surface on a cannula under a force pushing on the phaco tip toward the cannula. The first sealable surface can have a flat surface for pushing on the flat second sealable surface. Alternatively, the first sealable surface can have a convex surface for pushing on the flat second sealable surface. Alternatively, the first sealable surface can have a flat surface for pushing on the convex second sealable surface. Alternatively, the first sealable surface can have a seal element for mating with the second sealable surface. Alternatively, the second sealable surface can have a seal element for mating with the first sealable surface.

In FIG. 16B, operation 1620 forms a phaco tip, with the phaco tip having a first sealable surface configured to mate with a second sealable surface on a cannula under a force pushing on the phaco tip toward the cannula. The phaco tip can have a seal element configured to protrude into the cannula.

In FIG. 16C, operation 1640 forms an integrated cannula, with the integrated cannula having a cannula bonded with a phaco tip. The integrated cannula can have an aspiration passage running from a tip of the phaco tip to an end of the cannula.

### Interfaces of a Phaco Tip With a Sonotrode in a Phaco Handpiece

In addition to the coupling with the cannula, the phaco tip can also be coupled with the sonotrode. For example, the phaco tip can form a leak-free seal with the sonotrode, which is configured to prevent contamination of the sonotrode and to improve the ultrasonic energy transfer.

The leak-free seal of the phaco tip with the sonotrode can be a threaded seal, a snap seal, a press fit seal, or a magnetic seal. In addition to prevent a leakage of contamination fluid, the seal of the phaco tip with the sonotrode can provide a force acting on the seal of the phaco tip with the cannula, such as on the direct form fit seal, which can provide a pressure or a force on the form fit seal to maintain the seal structure. The coupling of the phaco tip and the sonotrode can be a solid-to-solid coupling, e.g., without a thick vibration absorbance material, to present loss of ultrasonic vibration power transferred from the sonotrode to the phaco tip.

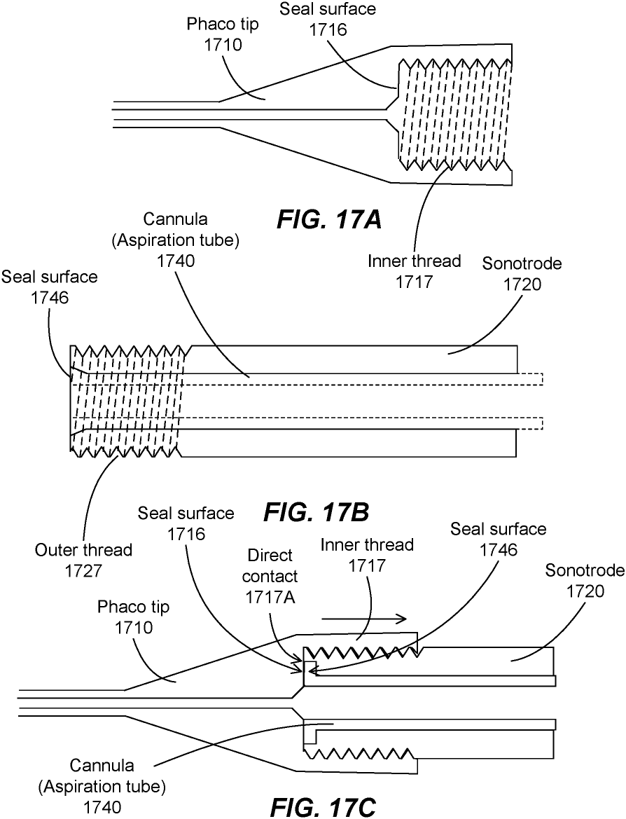

FIGS. 17A - 17C illustrate a threaded coupling between a phaco tip and a sonotrode according to some embodiments. A phaco tip 1710 can have a seal surface 1716 configured to form a seal with a seal surface 1746 of a cannula 1740, as discussed above. The phaco tip can include an inner thread 1717, which can be configured to be threaded with an outer thread 1727 of the sonotrode 1720.

In assembling, a cannula is assembled to the hollow portion of the ultrasonic drive. The phaco tip then can be threaded to the sonotrode, with the inner thread 1717 of the phaco tip threaded with the outer thread 1727 of the sonotrode. The threading action can be configured so that the seal surface 1716 of the phaco tip is mated with the seal surface 1746 of the cannula. Further, by threading to the sonotrode, the phaco tip can exert a pressure to the cannula to form the direct form fit seal between the phaco tip and the cannula.

In some embodiments, the coupling of the inner thread on the phaco tip threading on the outer thread on the sonotrode can be configured to provide a smooth path for the ultrasonic vibrations with a direct contact 1717A of the phaco tip with the sonotrode. The inner threading configuration of inner phaco tip thread coupling with outer threading of the sonotrode can thus be advantageous over the outer threading configuration of outer phaco tip thread coupling with inner sonotrode thread, since the surface of the sonotrode is in firm contact to surface of the tip and allows for good transmission of ultrasonic energy. In contrast, in the outer threading configuration, there is no direct contact since the ultrasonic vibration follows the spiral path of the thread. Consequently, there can be a large generation of heat, leading to potential damage of cornea tissues in contact with the phaco tip.

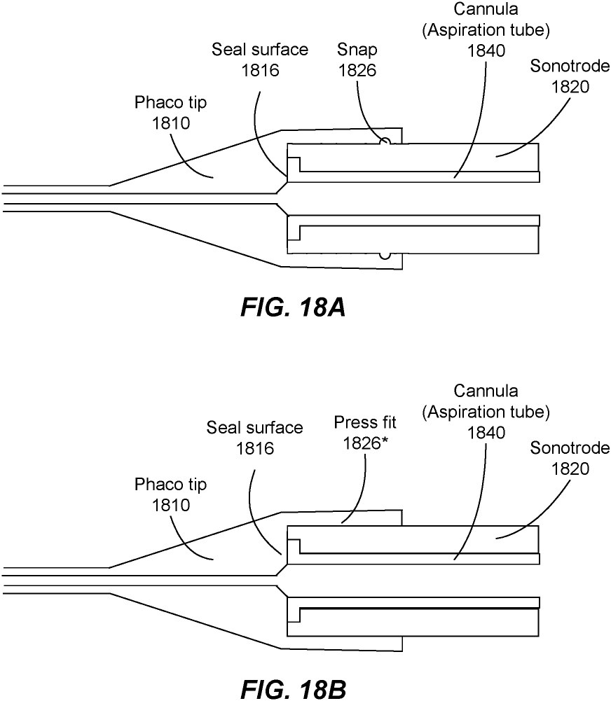

FIGS. 18A - 18B illustrate other coupling configurations between a phaco tip and a sonotrode according to some embodiments. In FIG. 18A, a snap fit 1826 on the sonotrode 1820 can be used to couple the phaco tip 1810 to the sonotrode 1820. The phaco tip still has a seal surface 1816 for making a seal with a mated seal surface on a cannula 1840. Similar to the thread coupling, the snap fit can also be configured to exert a force on the seal surfaces.

In FIG. 18B, a press fit 1826* between the phaco tip 1810 and the sonotrode 1820 can be used to couple the phaco tip 1810 to the sonotrode 1820, for example, by providing a progressively smaller diameter on the phaco tip or a progressively larger diameter on the sonotrode. The phaco tip still has a seal surface 1816 for making a seal with a mated seal surface on a cannula 1840.

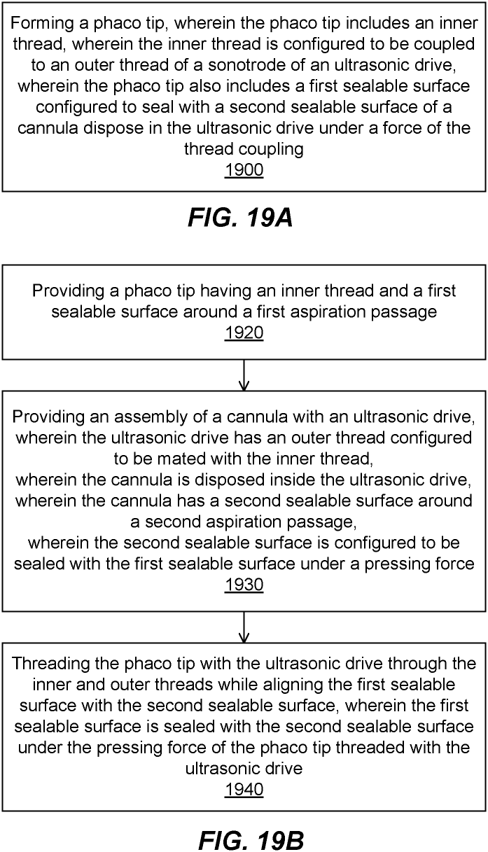

FIGS. 19A - 19B illustrate flow charts for forming a phaco tip to make a seal with a cannula and a sonotrode according to some embodiments. In FIG. 19A, operation 1900 forms a phaco tip, with the phaco tip including an inner thread, and with the inner thread configured to be coupled to an outer thread of a sonotrode of an ultrasonic drive. The phaco tip can also include a first sealable surface configured to seal with a second sealable surface of a cannula dispose in the ultrasonic drive under a force of the thread coupling.

In FIG. 19B, operation 1920 provides a phaco tip having an inner thread and a first sealable surface around a first aspiration passage. Operation 1930 provides an assembly of a cannula with an ultrasonic drive, with the ultrasonic drive having an outer thread configured to be mated with the inner thread of the phaco tip. The cannula can be disposed inside the ultrasonic drive. The cannula can have a second sealable surface around a second aspiration passage, with the second sealable surface configured to be sealed with the first sealable surface under a pressing force.

Operation 1940 threads the phaco tip with the ultrasonic drive through the inner and outer threads while aligning the first sealable surface with the second sealable surface, with the first sealable surface sealed with the second sealable surface under the pressing force of the phaco tip threaded with the ultrasonic drive.

### Interface Between Housing and Internal Components

A housing can be used to house the internal components of the phaco handpiece, e.g., the assembly of the phaco tip, the cannula, and the ultrasonic drive can be disposed in the housing. The housing can be configured to shield the ultrasonic drive from being exposed to the irrigation liquid. Further, the housing can be designed with an ergonomic style for added comfort, including for reducing physical stress and associated work injuries.

In some embodiments, the housing can be configured to seal around a base portion of the phaco tip at one end of the housing. For example, the housing can have an open end configured to fit around the base of the phaco tip.

In some embodiments, the housing can be configured to expose the ultrasonic drive at an opposite end of the housing. For example, the housing can have another open end configured to accept the ultrasonic drive and then remained open. Alternatively, the housing can have a housing end or cap configured to cover the exposed open end, to seal the end portion of the ultrasonic drive and also the cannula connector and the cable.

For assembly, the ultrasonic drive and the phaco tip can enter the housing from one open end and stop after a portion of the phaco tip protruded from the opposite open end with the housing configured to seal around the phaco tip at the opposite open end.

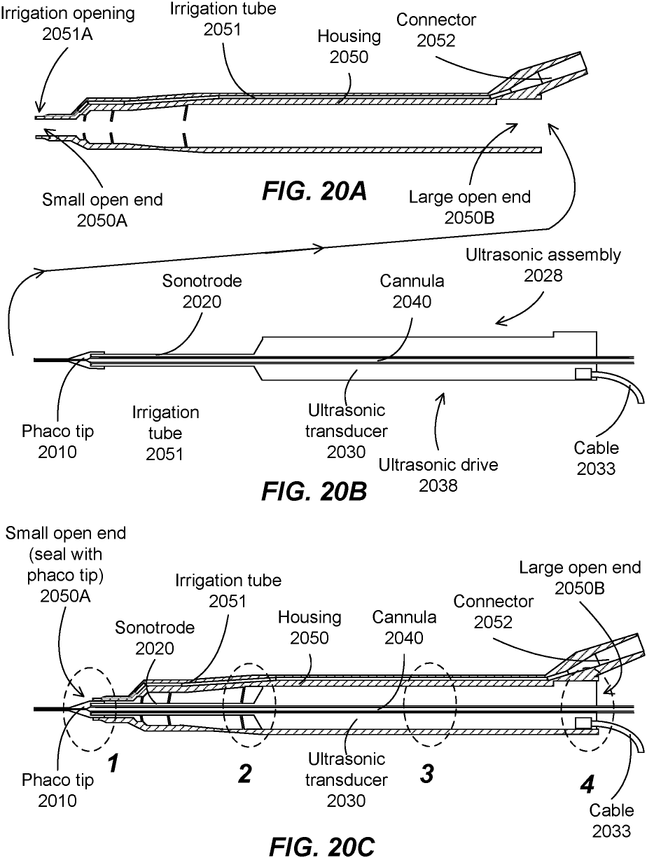

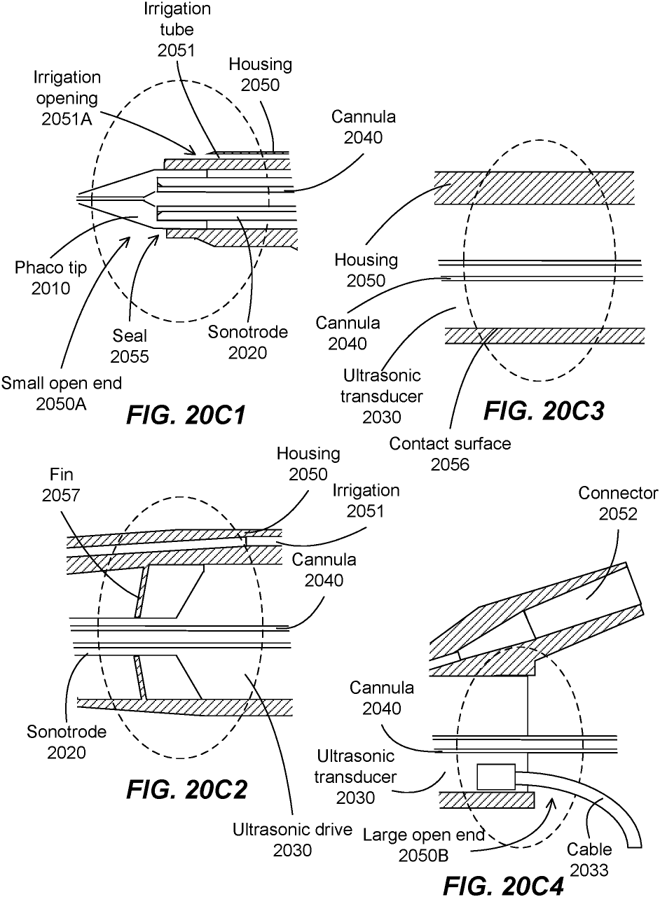

FIGS. 20A - 20C illustrate a configuration of a housing for a phaco handpiece according to some embodiments. FIG. 20A shows a housing 2050, which can have a tubular shape with a small open end 2050A at one end and a larger open end 2050B at an opposite end. An irrigation tube 2051 can be integrated to the housing, with one end forming a connector 2052 configured to be coupled to an irrigation system. The irrigation tube can run along the length of the housing, above, on, or under the housing surface, up to an opening 2051A at an opposite end to deliver the irrigation liquid to a phaco sleeve coupled to the housing at the small open end 2050A.

FIG. 20B shows an assembly 2028 of an ultrasonic drive 2038 having a sonotrode 2020 coupled to an ultrasonic transducer 2030. The assembly of the ultrasonic drive can include a phaco tip 2010 coupled to the sonotrode 2020 at one end and a cable 2033 coupled to the ultrasonic transducer 2030 at an opposite end. The assembly of the ultrasonic drive can also include a cannula 2040 assembled in a central hollow portion of an ultrasonic drive, with the cannula making a seal with the sonotrode 2020.

FIG. 20C shows the ultrasonic assembly 2028 assembled with the housing 2050. The ultrasonic assembly 2028 can be inserted into the housing from the large open end 2050B with the phaco tip 2010 entering the large open end first. The phaco tip can pass through the small open end 2050A and stop when the base portion of the phaco tip making a seal with the small open end 2050A, e.g., the phaco tip can be partially protruded from the housing. The ultrasonic assembly 2028 can have the end of the ultrasonic transducer exposed at the large open end 2050B, together with the protruded portion of the cannula and the cable 2033.

FIG. 20C1 - 20C4 show details of the housing assembly at portions 1 - 4, respectively. FIG. 20C1 shows an enlarged portion at the small open end 2050A of the housing 2050 at the portion labeled 1. The irrigation tube 2051 can have an opening 2051A at or near the small open end 2050A of the housing. The opening 2051A can be configured to be coupled with a phaco sleeve to deliver the irrigation liquid to an area near the phaco tip.

The phaco tip 2010 can protrude from the housing at the small open end 2050A and making a seal 2055 with the housing 2050. The seal 2055 can be configured to prevent the irrigation liquid from the irrigation opening 2051A from reaching the ultrasonic drive, such as to the sonotrode 2020, to contaminate the ultrasonic drive.

FIG. 20C2 shows an enlarged portion at a middle portion of the housing containing the sonotrode 2020 at the portion labeled 2. The housing can have one or more fins 2057 contacting the sonotrode. The fins can be flexible and have a small contact surface with the sonotrode, in order not to disturb the vibration of the sonotrode.

FIG. 20C3 shows an enlarged portion at a middle portion of the housing containing the ultrasonic transducer 2030 at the portion labeled 3. The housing can rest on the ultrasonic transducer 2030, or can have supports 2056 contacting the ultrasonic transducer. Since the ultrasonic transducer has low vibration amplitude, the contact of the housing with the ultrasonic transducer does not disturb the ultrasonic vibration.

FIG. 20C4 shows an enlarged portion at the large open end 2050B of the housing 2050 at the portion labeled 4. The end face of the ultrasonic transducer 2030 can be exposed, together with the protruded portion of the cannula to accept a connector for coupling to an aspiration pumping system and the cable 2033 to the driver system.

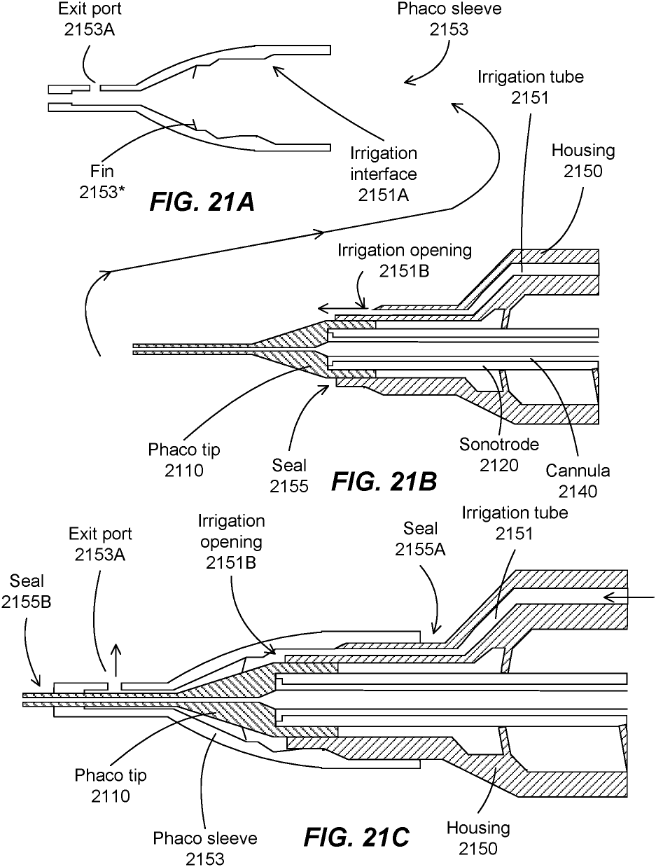

FIGS. 21A - 21C illustrate a phaco sleeve coupled to a housing according to some embodiments. FIG. 21A shows a phaco sleeve 2153 configured to be coupled with a housing 2150 at the small open end 2151A of the housing, e.g., at the location from which the phaco tip is protruded. The phaco sleeve can include one or more fins 2153* configured to contact the phaco tip for support. The fins can be flexible to not disturbing the vibrations of the phaco tip.

The phaco sleeve 2153 can include an irrigation interface 2151A, which is configured to mate with an irrigation opening 2151B at the end of an irrigation tube 2151 on the housing 2150. The phaco sleeve 2153 can include one or more exit ports 2153A configured to release an irrigation liquid received from the irrigation tube. Thus, the irrigation liquid can run along the irrigation tube 2151, passing through the interface with the phaco sleeve, e.g., coming through the opening 2151B to enter the phaco sleeve. The irrigation liquid can be configured to exit at the exit ports 2153A, for example, by sealing off other possible exit paths, such a sealing 2155B the phaco sleeve with the tip of the phaco tip, and sealing 2155A the phaco sleeve with the housing. The seal 2155A with the housing can be leak-free, to prevent the contamination of the irrigation liquid with other parts of the phaco handpiece. The seal 2155B with the phaco tip is not required to be leak-free, e.g., since the leaked irrigation liquid would not contaminate the phaco handpiece.

FIG. 21B shows a portion of the housing 2150 assembled with a phaco tip 2110, the cannula 2140, and the sonotrode 2120. The phaco tip 2110 is assembled to the housing with a leak-free seal 2155, which is configured to prevent the irrigation liquid passing through the opening 2151B to contaminate the sonotrode. The assembled housing can be inserted to the sleeve 2153, so that one end of the sleeve makes a seal 2155B on the tip of the phaco tip, and the opposite end of the sleeve makes a seal 2155A with the housing. The assembling process can be configured to enable the irrigation interface 2151A on the sleeve matches with the opening 2151B on the housing, so that the irrigation liquid can enter the sleeve.

FIG. 21C shows an assembling of the phaco sleeve with the housing together with the phaco tip. The irrigation liquid can pass through the irrigation opening 2151B to enter the sleeve 2153. Since the sleeve is configured to make a seal 2155A with the housing and a seal 2155B with the phaco tip, the irrigation liquid can only exit through the exit port 2153A.

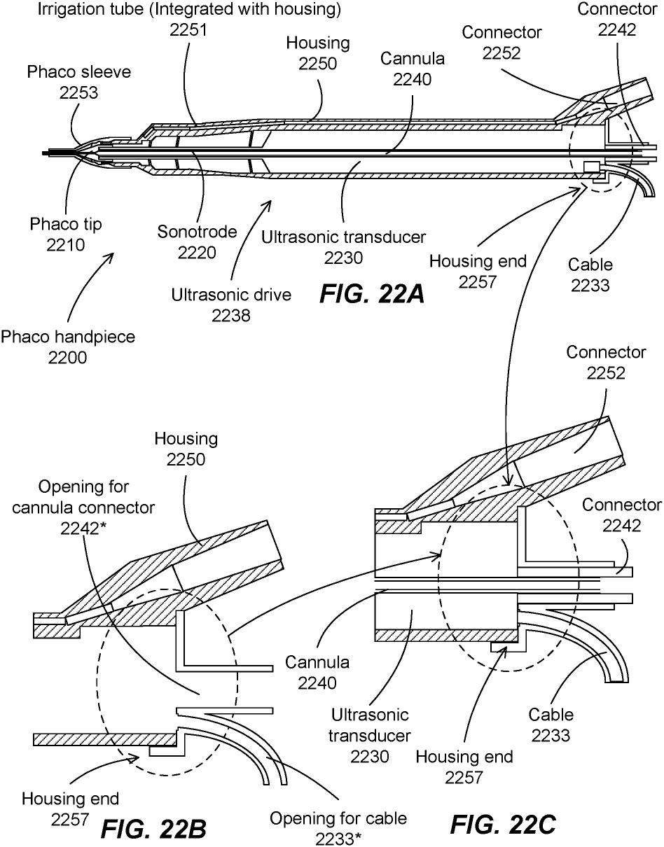

FIGS. 22A - 22C illustrate a housing end for a housing according to some embodiments. FIG. 22A shows a complete assembling of a phaco handpiece, including an optional housing end 2250A to seal the end portion of the ultrasonic drive 2238, the cannula connector 2242, and the cable 2233. After assembling the ultrasonic drive with the housing, the end portion of the ultrasonic drive can be exposed, and thus can be optionally covered with the housing end 2257.

FIG. 22B shows a housing end 2157 coupled to the housing without the ultrasonic drive. The housing end can be configured to be coupled to the open end of the housing. The housing end can have an opening 2242* for the cannula connector, and another opening for the cable 2233*. FIG. 22C shows the housing end 2157 coupled to the housing with the ultrasonic drive. The opening 2242* for the cannula connector can be sealed with the cannula connector 2242. The opening for the cable 2233* can be sealed with the cable 2233.

In some embodiments, the present invention discloses a phaco handpiece 2200, which can be a medical handpiece configured to perform phacoemulsification surgery. The phaco handpiece 2200 can include an ultrasonic drive 2238 having a sonotrode 2220 coupled to an ultrasonic transducer 2230, a cannula 2240 detachably coupled to a channel through the sonotrode 2220 and the ultrasonic transducer 2230 of the ultrasonic drive 2238, a phaco tip 2210 coupled to the end of the sonotrode 2220, with the phaco tip having an aspiration passage configured to form a fluid communication with the cannula.

The phaco handpiece 2200 can include a housing 2250 configured to house the ultrasonic assembly, which includes the ultrasonic drive having the cannula assembled within and the phaco tip coupled to the sonotrode. A phaco sleeve 2253 can further be coupled to the housing to cover the tip portion of the phaco tip.

The housing can include an irrigation tube 2251 configured to deliver an irrigation liquid. The irrigation liquid can be configured to be delivered to the phaco sleeve, for example, through an irrigation interface with the phaco sleeve. The housing can include a connector 2252 coupling to the irrigation tube for coupling to an irrigation system. A connector 2242 can be configured to be coupled to the protruded portion of the cannula for coupling to an aspiration pumping system.

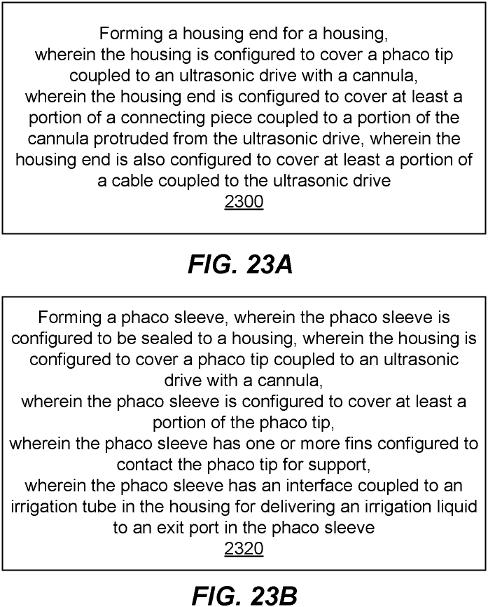

FIGS. 23A - 23B illustrate flow charts for forming elements of a phaco handpiece according to some embodiments. In FIG. 23A, operation 2300 forms a housing end for a housing, with the housing configured to cover a phaco tip coupled to an ultrasonic drive with a cannula. The housing end is configured to cover at least a portion of a connecting piece coupled to a portion of the cannula protruded from the ultrasonic drive. The housing end is also configured to cover at least a portion of a cable coupled to the ultrasonic drive.

In FIG. 23B, operation 2320 forms a phaco sleeve, with the phaco sleeve configured to be sealed to a housing, and with the housing configured to cover a phaco tip coupled to an ultrasonic drive with a cannula. The phaco sleeve is configured to cover at least a portion of the phaco tip. The phaco sleeve has one or more fins configured to contact the phaco tip for support. The phaco sleeve has an interface coupled to an irrigation tube in the housing for delivering an irrigation liquid to an exit port in the phaco sleeve.

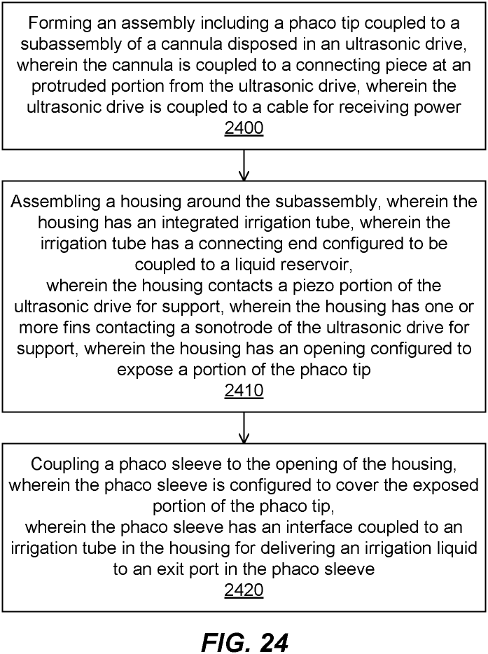

FIG. 24 illustrates a flow chart for assembling a phaco handpiece according to some embodiments. Operation 2400 forms an assembly including a phaco tip coupled to a subassembly of a cannula disposed in an ultrasonic drive, with the cannula coupled to a connecting piece at an protruded portion from the ultrasonic drive, and with the ultrasonic drive coupled to a cable for receiving power.

Operation 2410 assembles a housing around the subassembly, with the housing having an integrated irrigation tube, and with the irrigation tube having a connecting end configured to be coupled to a liquid reservoir. The housing contacts a piezo portion of the ultrasonic drive for support. The housing has one or more fins contacting a sonotrode of the ultrasonic drive for support. The housing has an opening configured to expose a portion of the phaco tip.

Operation 2420 couples a phaco sleeve to the opening of the housing, with the phaco sleeve configured to cover the exposed portion of the phaco tip. The phaco sleeve has an interface coupled to an irrigation tube in the housing for delivering an irrigation liquid to an exit port in the phaco sleeve.

### On-Off Haptic Feedback

In some embodiments, the phaco handpiece can include a haptic feedback to provide the surgeon with information regarding the operating conditions of the phaco handpiece or status of the cataract. Ophthalmic surgical system typically includes a hand-held medical tool, such as a handpiece with a tip. Operation of the handpiece requires controls of settings or functions needed for the surgical procedure, such as turning on and setting an ultrasonic drive system for emulsifying eye tissue, regulating flows from an irrigation source for irrigating the eye with a saline solution, and regulating suctions from an aspiration pump for aspirating the emulsified eye tissue from the eye.

During the surgery, such as an ophthalmic surgery, it is important that the surgeon is provided with information regarding various surgical functions and parameters. The haptic feedback can allow the surgeon to monitor surgical conditions during surgery without any additional movements of the surgeon or without distracting the surgeon attention, such as a display, flashing lights, voice information, beeps or buzzers. In the phaco handpiece haptic feedback, the information is conveyed to the surgeon in form of vibration patterns or waveforms through the surgeon hand holding the phaco handpiece. A haptic feedback system can allow the surgeon to perform the surgery with no need to worry about an abrupt audio tone or a distracting visual indicator.

In some embodiments, a haptic feedback can be incorporated in the phaco handpiece to provide the surgeon with information regarding the working condition of the phaco handpiece, e.g., whether or not ultrasonic vibrations are present at the phaco tip. The haptic feedback can be located in the phaco handpiece, with a vibration plate on an outer surface of the phaco handpiece. The haptic feedback mechanism can be configured to receive an indication that the ultrasonic drive is in operation, and can also be configured to provide vibration energy to the phaco handpiece to be felt by the surgeon during the surgical operation. Since during the surgical operation, the focus of the surgeon should be on the surgical area, the haptic feedback at the handpiece would be better suited as compared to a remote console such as a foot switch.

The haptic feedback can include a sensor, such as a vibration coupler, such as a partial metal ring, coupled to the sonotrode. The haptic feedback can include a receiver, such as a feedback indicator, such as a metal plate, on an outer surface of the housing, e.g., at a location contacting the surgeon hand, to provide a haptic feedback to the surgeon. A solid connection can be provided between the sensor and the receiver, which can allow the vibration to reach the receiver when the sensor senses vibration at the sonotrode. The solid connection can be configured to optimize the haptic feedback, e.g., to provide a vibration at the receiver with an appropriate amplitude comfortable to the surgeon. Alternatively, an adjustable connection can be provided to allow the surgeon to change the levels of the haptic feedback.

In operation, when the phaco handpiece is turned on, e.g., when the ultrasonic drive receives power from the driver system to generate the vibration at the sonotrode, the sensor can also be vibrated. The vibration from the sensor is communicated to the receiver, through the solid connection. Upon feeling the vibration in the hand, the surgeon can recognize that the phaco handpiece is on. When the phaco handpiece is turned off, there is no vibration at the sonotrode, which leads to no vibration at the receiver. The surgeon can recognize that the phaco handpiece is off.

The haptic feedback can be particular useful for a disposable phaco handpiece having a single use plastic housing, since the inexpensive plastic housing might not conduct vibration transfer well. Further, minimum or no vibration transfer to the housing can be useful to prevent loss of vibration energy generated from the ultrasonic drive. The haptic feedback can receive minimum vibration transfer from the ultrasonic drive, thus can provide feedback to the surgeon without or with minimum disturbing the vibration transfer.

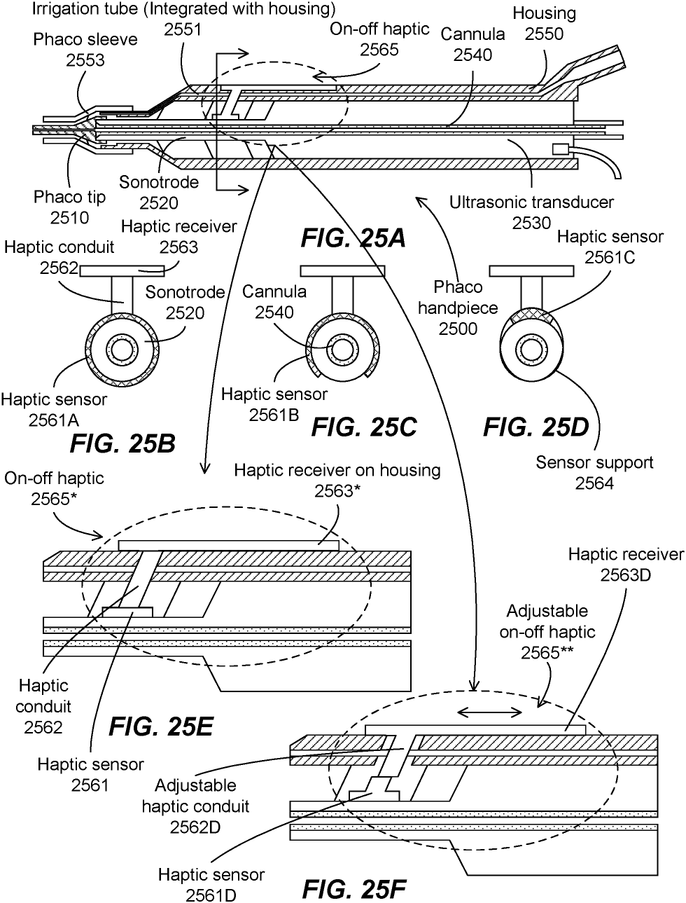

FIGS. 25A - 25F illustrate haptic feedback configurations for a phaco handpiece according to some embodiments. In FIG. 25A, a phaco handpiece 2500 can include an ultrasonic drive having an ultrasonic transducer 2530 driving a sonotrode 2520. A phaco tip 2510 is coupled to the sonotrode 2520 to receive the ultrasonic vibration for emulsifying the cataract at the tip of the phaco tip. The ultrasonic drive can be disposed in a housing 2550, which is coupled to a phaco sleeve 2553 for covering the phaco tip. An irrigation tube 2551 can be integrated with the housing 2550 to deliver an irrigation liquid to the surgery site.

In some embodiments, the phaco handpiece can be a component-wise disposable phaco handpiece, with the ultrasonic drive configured to be reusable, the housing and the phaco sleeve configured to be disposable, and other components configured to be either disposable or reusable. The disposable housing can be formed by a polymer material, such as plastic, to be low cost with various ergonomic shapes for different surgeon hands.

With the plastic housing, there can be low vibration lost, thus the phaco handpiece can be more energy efficient. However, with low vibrations at the housing, the surgeon can have difficulty in knowing whether or not the ultrasonic drive is in operation through the phaco handpiece.

In some embodiments, an on-off haptic feedback can include a fingertip plate attached to the housing, and connected to a vibration sensor, for example, through a vibration conduit, e.g., materials that have low resistance to vibration. The finger tip plate can provide a tactile feedback to the surgeon regarding some functions of the phaco handpiece, such as whether or not there are ultrasonic vibrations at the phaco tip. The finger tip vibrations can provide the surgeon with a real time feel for the ultrasonic energy generated by the ultrasonic transducer.

An on-off haptic feedback 2565 can be coupled to the housing. The on-off haptic feedback can include a haptic receiver 2563, such as a metal plate configured for receiving ultrasonic vibrations. The haptic receiver 2563 is coupled to an outer surface of the housing 2550, such as embedded in the plastic shell of the housing, e.g., having a top surface flushed with the surrounding plastic housing surface. Other configurations can be used, such as the haptic receiver is disposed on the housing surface, or partly embedded in the housing. The haptic receiver is placed at a location that is likely to be contacted by the surgeon hand, e.g., a thumb or a finger tip, when holding the phaco handpiece.

The on-off haptic feedback 2565 can include a haptic sensor 2561 configured to sense the vibrations from the ultrasonic drive, and to transfer the vibration to the haptic receiver 2563 through a haptic conduit 2562. The haptic sensor 2561 can include a high vibration material, e.g., having low vibration resistance, which is coupled to the ultrasonic drive, such as coupled to the sonotrode or to the transducer. By contacting the ultrasonic drive the haptic sensor can sense the vibration and can vibrate in response to the vibration generated by together with the ultrasonic drive. The haptic conduit 2562 and the haptic receiver 2563 can also include a high vibration material, to transfer the vibration sensed by the haptic sensor.

In operation, when the ultrasonic drive is turned on, the vibration generated by the ultrasonic drive can be sensed by the haptic sensor. The vibration sensed by the haptic sensor can be transferred to the haptic conduit, and then to the haptic receiver. When the ultrasonic drive is turned off, there is no vibration at the haptic receiver.

FIG. 25B shows a haptic sensor 2561A in the form of a clip ring attached to the sonotrode 2520. The haptic sensor can be coupled to a haptic conduit 2562, and then to the haptic receiver 2563. FIG. 25C shows a haptic sensor 2561B in the form of a partial ring clipped on the sonotrode 2520. The haptic sensor can be coupled to a haptic conduit 2562, and then to the haptic receiver 2563. FIG. 25D shows a haptic sensor 2561C in the form of an arc or a plate attached to the sonotrode 2520 by a sensor support 2564, such as a clip or a ring. The haptic sensor can be coupled to a haptic conduit 2562, and then to the haptic receiver 2563.

The haptic sensor can be attached to the sonotrode at locations that can least interfere with the operation of the ultrasonic drive, such as at a base of the sonotrode near the ultrasonic transducer. Alternatively, the haptic sensor can be coupled to the ultrasonic transducer, such as at a location near the piezo elements. In some embodiments, the haptic sensor can be designed as an integral part of the ultrasonic drive, such as the sensor is configured as a part of the sonotrode, in order to optimize the ultrasonic power delivered to the sonotrode and ultimately to the phaco tip.

FIG. 25E shows an alternate on-off haptic feedback 2565* with the haptic receiver 2563* disposed on the surface of the plastic housing, e.g., protruded from the surface of the housing. The protrusion can be small, such as less than 2 mm or less than 1 mm, and can serve to let the surgeon know the location of the on-off haptic feedback.

In some embodiments, the on-off haptic feedback can include a control to adjust the level of vibration on the haptic receiver. For example, the haptic conduit between the sensor and the receiver can be controlled, e.g., adjusting its vibration resistance, so that the amount of vibration transferred from the haptic sensor to the haptic receiver can be suitable to the surgeon.

FIG. 25F shows an adjustable on-off hepatic feedback 2565**, e.g., an on-off haptic feedback with a control to vary the level of vibration on the receiver 2563D. The adjustable on-off hepatic feedback 2565** can include a haptic sensor 2561D coupled to a vibration portion of the ultrasonic drive, such as coupled to the sonotrode or to the piezo element of the ultrasonic transducer. The haptic feedback 2565** can include two portions haptic conduit 2562D that can movably coupled to each other. One portion of the haptic conduit is coupled to the haptic sensor. The other portion of the haptic conduit is coupled to the haptic receiver. By changing the amount of contact between the two portions, such as sliding the receiver with respect to the sensor, the vibration level received by the receiver can be adjusted.

In an alternative configuration, the on-off haptic feedback can be independent of the vibration generated by the ultrasonic drive, e.g., there can be no sensor coupled to the sonotrode . By not using a haptic sensor coupled to the sonotrode, the haptic feedback can eliminate any vibration loss from the ultrasonic drive. The alternate on-off haptic feedback can include a vibration unit coupled to housing and controlled by the electrical power supplied to the ultrasonic drive. For example, the vibration unit can be coupled to a plate embedded or disposed on the housing, with the plate located at likely finger tip locations when the surgeon uses the phaco handpiece.

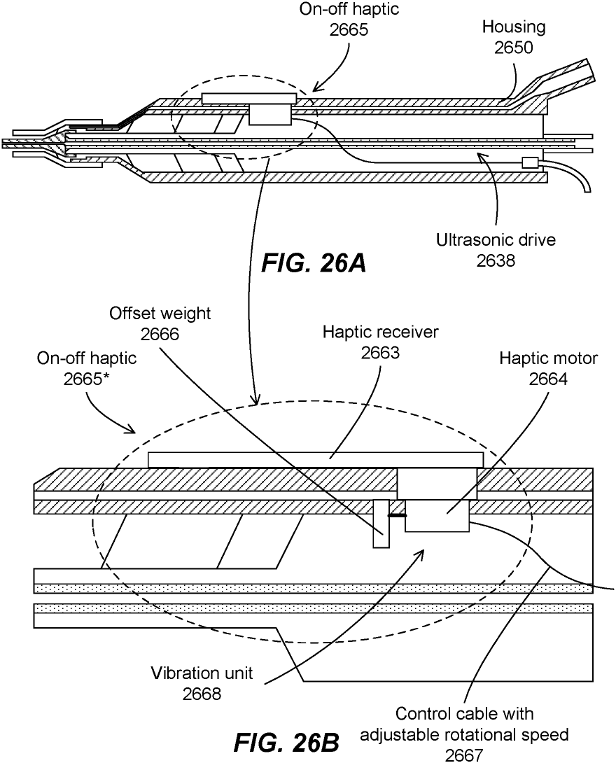

FIGS. 26A - 26B illustrate another haptic feedback configuration for a phaco handpiece according to some embodiments. A haptic feedback 2665 having its own vibration unit 2668 can be configured to vibrate when the ultrasonic drive 2638 is turned on.

The vibration unit 2668 can be coupled to an outer surface of the housing, such as coupled to a haptic receiver 2663, which can be a low vibration resistance plate coupled to an outer surface of the plastic housing 2650. The haptic receiver can provide a tactile indicator through the vibration unit 2668, which, for example, can include a motor 2664 and an off-center or offset weight 2666. The motor 2664 can be configured to be electronically coupled to a controller through a control cable 2667 to receive a signal from the controller when the ultrasonic drive is turned on. When receiving a signal from the controller indicating that the ultrasonic drive is powered, the motor can rotate the off-center weight. The off-center weight can be configured to vibrate when rotating, e.g., when rotating about a rotation axis 260, the unbalanced off-center weight can cause the off-center weight to also move with the shifting of the center of mass. Other vibration mechanism can be used, such as a rotating fin engaging or hitting the receiver plate.

The on-off haptic feedback can have the vibration unit 2668 coupled to a receiver plate 2663 to cause the receiver plate to vibrate. For example, the receiver plate can be coupled to the housing 2650 at the finger tip location of the surgeon. Power to the vibration unit, e.g., to the motor 2664, can be provided by the driver system of the ultrasonic driver after the ultrasonic driver is turned on. For example, the vibration unit can receive power concurrently as the ultrasonic drive, thus, then the ultrasonic drive is turned on, the vibration unit also vibrates, which gives the surgeon a haptic feedback indicating that the ultrasonic drive is on.

Alternatively, power to the vibration unit can be coupled to a current sensing circuit which is configured to sense a current drawn by the piezo elements of the ultrasonic transducer. Thus, the vibration unit can be turned on when the current sensing circuit measures an appropriate current supplied to the ultrasonic transducer. The current sensing circuit can ensure that the vibration unit is linked to the vibration generation of the ultrasonic transducer, instead of only linked to the power supplied to the ultrasonic transducer. For example, if the ultrasonic transducer is broken or damaged, the current sensed by the current sensing circuit cannot reach a current threshold, and the vibration unit does not vibrate, indicating that the ultrasonic transducer does not generate ultrasonic vibration, even there can be power supplied to the ultrasonic transducer.

In some embodiments, the power supplied to the vibration unit, e.g., to the vibration motor, can be adjustable, e.g., high power for a strong vibration or low power for a weak vibration, depending on a preference of the surgeon. A power adjustment circuit can be coupled to the vibration motor, and can be controlled by the surgeon to adjust the level of vibration of the haptic feedback by adjusting the power applied to the vibration unit.

An advantage of the haptic feedback using the vibration unit is the decoupling of the ultrasonic drive with the haptic feedback. The haptic feedback does not disturb the ultrasonic vibration transfer generated by the ultrasonic drive.

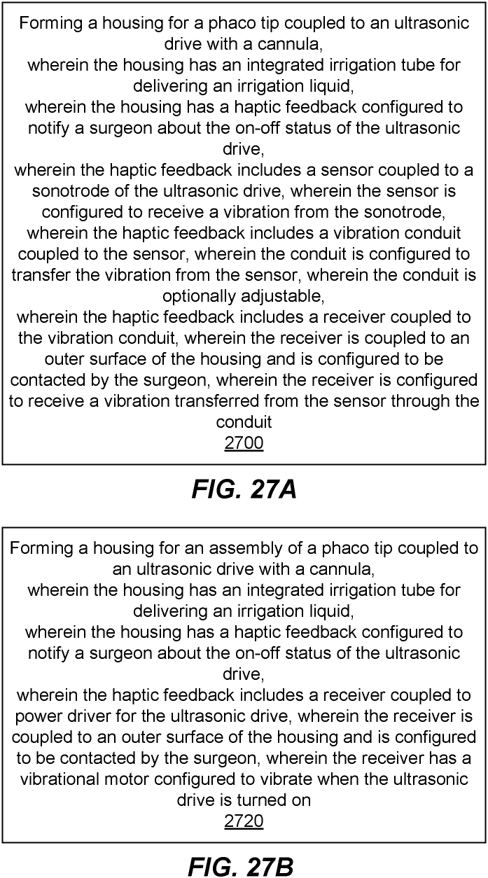

FIGS. 27A - 27B illustrate flow charts for forming a phaco handpiece having an on-off haptic feedback according to some embodiments. In FIG. 27A, operation 2700 forms a housing for a phaco tip coupled to an ultrasonic drive with a cannula. The housing can have an integrated irrigation tube for delivering an irrigation liquid. The housing can have a haptic feedback configured to notify a surgeon about the on-off status of the ultrasonic drive.

The haptic feedback can include a sensor coupled to a sonotrode of the ultrasonic drive, with the sensor configured to receive a vibration from the sonotrode. The haptic feedback can include a vibration conduit coupled to the sensor, with the conduit configured to transfer the vibration from the sensor, and with the conduction of the conduit being optionally adjustable.

The haptic feedback can include a receiver coupled to the vibration conduit, with the receiver coupled to an outer surface of the housing and configured to be contacted by the surgeon. The receiver can be configured to receive a vibration transferred from the sensor through the conduit.

In FIG. 27B, operation 2700 forms a housing for an assembly of a phaco tip coupled to an ultrasonic drive with a cannula. The housing can have an integrated irrigation tube for delivering an irrigation liquid. The housing can have a haptic feedback configured to notify a surgeon about the on-off status of the ultrasonic drive. The haptic feedback can include a receiver coupled to power driver for the ultrasonic drive, with the receiver coupled to an outer surface of the housing and configured to be contacted by the surgeon. The receiver can have a vibration motor configured to vibrate when the ultrasonic drive is turned on

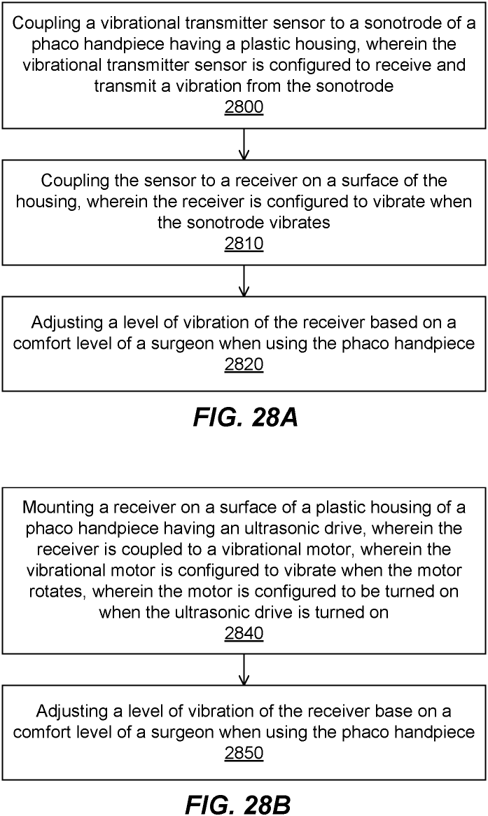

FIGS. 28A - 28B illustrate flow charts for operating a phaco handpiece having an on-off haptic feedback according to some embodiments. In FIG. 28A, operation 2800 couples a vibration transmitter sensor to a sonotrode of a phaco handpiece having a plastic housing, with the vibration transmitter sensor configured to receive and transmit a vibration from the sonotrode. Operation 2810 couples the sensor to a receiver on a surface of the housing, with the receiver configured to vibrate when the sonotrode vibrates. Operation 2820 adjusts a level of vibration of the receiver based on a comfort level of a surgeon when using the phaco handpiece.

In FIG. 28B, operation 2840 mounts a receiver on a surface of a plastic housing of a phaco handpiece having an ultrasonic drive, with the receiver coupled to a vibration motor, with the vibration motor configured to vibrate when the motor rotates, and with the motor configured to be turned on when the ultrasonic drive is turned on. Operation 2850 adjusts a level of vibration of the receiver base on a comfort level of a surgeon when using the phaco handpiece.

In some embodiments, the present invention discloses a phaco handpiece, e.g., a medical handpiece configured to perform phaco emulsification surgery. The medical handpiece can include a haptic feedback configured to provide an operation indication of the medical handpiece, with the haptic feedback including a vibrationable plate coupled to a vibration coupler with the vibration coupler coupled to the sonotrode. The vibrationable plate can be disposed on an outer surface of the housing, and configured to let a surgeon holding the medical handpiece to receive a vibration indicating that the medical handpiece is operating.

In some embodiments, the haptic feedback can include a vibrationable plate coupled to a vibrating motor, with the vibrating motor electrically coupled to a power line configured to power the ultrasonic transducer. The vibrationable plate can be disposed on an outer surface of the housing, and configured to let a surgeon holding the medical handpiece to receive a vibration indicating that the medical handpiece is operating.

In some embodiments, the haptic feedback can include an adjustable component configured to adjust a vibration level of the vibrationable plate based on a comfort level of a surgeon operating the medical handpiece. The vibrationable plate can be at least partially embedded in the housing. The vibrationable plate can include a metallic material coupled to a housing formed from a polymer material.

### Material Characteristic Haptic Feedback

In some embodiments, the phaco handpiece can include a material haptic feedback, such as a cataract detection, a density detection, or detections of different layers in the eye, such as a detection of the lens and the posterior capsule. The material haptic feedback can provide information about the structure of the eye, including the locations of different layers of the eye, such as the location of the posterior capsule when the phaco handpiece contacts the lens for emulsification to prevent damaging, such as preventing the emulsification of the posterior capsule. The material haptic feedback can provide density information based on the force encountered by the phaco tip, with the eye structures determined from the density differences. For example, the cataract lens can have a higher density than the posterior capsule, due to the hardening of the lens. By detecting the density difference, the surgeon can know the location of the posterior capsule, and thus can maneuver the phaco tip away from the posterior capsule to avoid damaging or destroying the posterior capsule

The material haptic feedback can include an ultrasonic sensor configured to measure the ultrasonic disturbance on the ambient due to the ultrasonic vibration generated by the ultrasonic drive. The ultrasonic sensor can senses an ultrasonic field generated through the vibrations at the phaco tip travelling through the ambient between the phaco tip and the ultrasonic sensor.

The measured ultrasonic field at the ultrasonic sensor can be provided to a controller, which then can process the data to generate a parameter related to the ambient surrounding the phaco tip at the surgery site, such as hardness or softness of the cataract. The measured data can be calibrated, for example, by calculating the field difference with and without the cataract ambient at the surgery site.

A vibration unit can be coupled to an outer surface of the housing and is configured to receive information from the controller. Depending on the data processed from the acoustic sensor, the vibration unit can vibrate at different rotational speeds or at different pulse sequences, to let the surgeon know about the structure and characteristics of the cataract.

Alternatively, the material haptic feedback can further include an additional ultrasonic transducer configured to generate a different ultrasonic field. The different ultrasonic field can be selected to provide better information on the structure and characteristics of the fluid ambient at the surgery site.

In some embodiments, the phaco handpiece can be configured to vibrate at two ultrasonic frequencies, with a first frequency configured to emulsify the cataract and the second frequency configured to diagnose the cataract. The frequencies can be selected based on the structure and characteristics of the cataract, but with different objectives. For example, the first frequency can be selected based on the emulsification characteristics of the cataract, for example, between 26 kHz and 50 kHz. The second frequency can be selected based on the absorption or scattering characteristics of the cataract, for example, between 5 MHz and 10 MHz. There can be two transducers, or one transducer with two sets of piezo elements to generate ultrasonic vibrations at different frequencies. In addition, a selector can be included at the phaco handpiece to allow the surgeon to switch frequencies during the surgical operation.

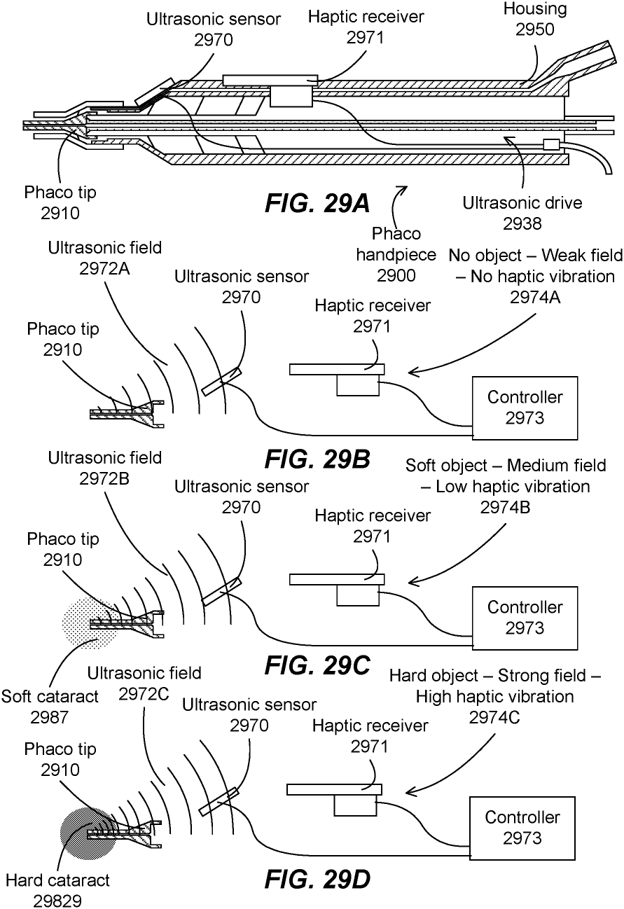

FIGS. 29A - 29D illustrate a phaco handpiece having an ultrasonic haptic feedback according to some embodiments. In FIG. 29A, a phaco handpiece 2900 can have an ultrasonic drive 2938 coupled to a phaco tip for emulsifying cataract materials in an eye site. The surgical operation, including the emulsification of the cataract materials, can depend on the characteristics of the cataract, such as how hard or how soft the cataract is.

In some embodiments, the ultrasonic haptic feedback can assess the ultrasonic field passing through the cataract materials to provide information of the cataract characteristics, including the hardness of the cataract. The information can assist the surgeon to make on-the-field decision during the surgical operation.

The ultrasonic haptic feedback can measure the ultrasonic field generated by the ultrasonic drive in the phaco handpiece to assess the disturbance of the ultrasonic field in the presence of the cataract. For example, the measured ultrasonic field during the surgery can be compared with a baseline ultrasonic field, e.g., an ultrasonic field measured without the cataract. The difference between the two ultrasonic fields can provide indications of the cataract characteristics, which can assist the surgeon during the operation.

The ultrasonic haptic feedback can include an ultrasonic sensor 2970, which can be configured to measure the ultrasonic field around the phaco tip 2910. The measured data can be provided to a controller 2973, to assess the cataract characteristics. The controller then can send vibration signals to a haptic receiver 2971 located on a surface of the phaco housing 2950.

The haptic receiver can include a vibration unit coupled to a receiver plate to cause the receiver plate to vibrate. The vibration unit can include a motor and an off-center or offset weight. Vibration signals sent by the controller to the vibration unit, e.g., to the motor, to cause the motor to rotate, which leads to the vibration unit to vibrate. The levels of vibration can be based on the characteristics of the cataract determined by the controller based on the measured ultrasonic field.

FIGS. 29B - 29D show a calibration process for the assessment of the cataract characteristics. In FIG. 29B, the ultrasonic sensor 2970 can be used to measure the ultrasonic field 2972A when the phaco tip 2910 is outside of the surgical site, e.g., at an air ambient for example. The measured ultrasonic field 2972A can be used as a reference field, e.g., establishing a base line ultrasonic field. Under this reference field, the controller 2973 can send no vibration signals 2974A to the haptic receiver 2971, indicating that there is no cataract.

In FIG. 29C, the ultrasonic sensor 2970 can be used to measure the ultrasonic field 2972B when the phaco tip 2910 is at a surgical site in which a soft cataract 2987 is known. The measured ultrasonic field 2972B can be used as a characteristic field for soft cataract, e.g., establishing a data point for soft cataract characteristics. Under this soft cataract characteristic field, the controller 2973 can send low vibration signals 2974B to the haptic receiver 2971, indicating that the cataract includes a soft material.

In FIG. 29D, the ultrasonic sensor 2970 can be used to measure the ultrasonic field 2972C when the phaco tip 2910 is at a surgical site in which a hard cataract 2988 is known. The measured ultrasonic field 2972C can be used as a characteristic field for hard cataract, e.g., establishing a data point for hard cataract characteristics. Under this hard cataract characteristic field, the controller 2973 can send high vibration signals 2974C to the haptic receiver 2971, indicating that the cataract includes a hard material.

After the calibration of measuring the ambient, a soft cataract and a hard cataract, the controller can have data point for relating a new ultrasonic field to the hardness of the cataract, and can send vibration signals to the haptic receiver according to the assessed characteristics.

In some embodiments, the cataract characteristic haptic feedback can include a second transducer specifically optimized for detecting the cataract characteristics. Since the ultrasonic frequency of a phaco handpiece is optimized for emulsifying the cataract, a different frequency can be more suited to detecting the cataract characteristics. Thus, the cataract characteristic haptic feedback can include a transducer configured to generate a new frequency different from the emulsified frequency, and a sensor configured to detect the new frequency. The measured field from the sensor can be sent to the controller for assessing the cataract characteristics, and vibration signals can be sent to the haptic receiver to let the surgeon know about the cataract, based on tactile feedback. In some embodiments, the new frequency can be any frequency, ranging from acoustic frequencies (e.g., less than 20 kHz) to ultrasonic frequencies (greater than 20 kHz) and beyond. For example, the new frequency can be a diagnostic frequency used in medical imaging, which is between 5 and 10 MHz.

For using an additional frequency for characterizing the cataract, the ultrasonic sensor can include a circuit to filter out the signals from the ultrasonic frequency generated for emulsification.

In some embodiments, the cataract characteristic haptic feedback can include a force transducer coupled to the phaco handpiece for measuring a force encountered by the phaco tip. The force transducer can be configured to measure the force that the phaco tip applies to the cataract during the emulsification process. This can be done by measuring the force that the cataract applies to the phaco tip, since these two forces are equal and of opposite directions.

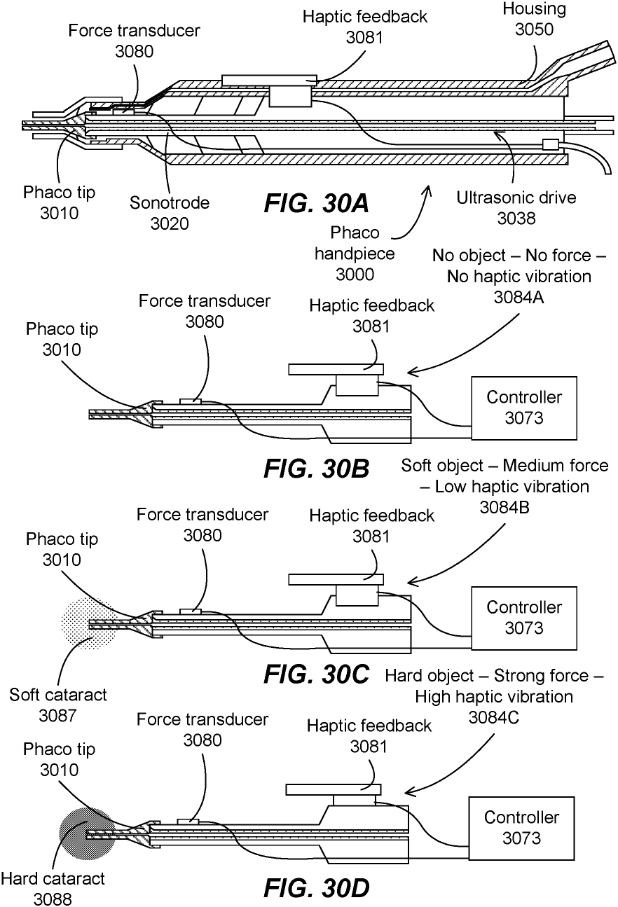

FIGS. 30A - 30D illustrate a phaco handpiece having a force transducer haptic feedback according to some embodiments. The haptic force feedback 3081 can include a force sensor 3080, such as a force transducer, coupled to the ultrasonic drive 3038, such as to the phaco tip 3010, the sonotrode 3020 or the ultrasonic transducer, to measure a force on the phaco tip. The force data can be supplied to the vibration unit to provide a haptic force feedback to the surgeon.

The force transducer can be a six-axis force transducer, which can measure forces on three linear axes and optional torques on three rotational axes. The forces can be measured and analyzed, with calibration using a controller 3073. The force transducer can be attached to the ultrasonic drive, such as to the sonotrode or to the ultrasonic transducer at one side. At an opposite side, the force transducer can be protected by the housing of the phaco handpiece. The housing can be configured to be surrounding the force transducer, to prevent other objects from touching the force transducer or to the ultrasonic drive.

The housing can prevent the force transducer from being touched by other objects. Thus, the only forces acting on the phaco tip is the measured force, the contact force, and the probe weight, ignoring the force due to acceleration since the phaco handpiece moves slowly. The force transducer can be connected by a cable to a controller 3073, for example, in a driver system configured to provide ultrasonic signals to the ultrasonic drive of the phaco handpiece. The force transducer can include a strain gauge, which can generate an analog voltage.

The force haptic feedback can include a force sensor 3080 configured to measure the force experienced by the phaco tip 3010. The measured force can be provided to a controller 3073 to assess the cataract characteristics. The controller then can send vibration signals to a haptic receiver 3071 located on a surface of the phaco housing 3050.

The haptic receiver can include a vibration unit coupled to a receiver plate to cause the receiver plate to vibrate. The vibration unit can include a motor and an off-center or offset weight. Vibration signals sent by the controller to the vibration unit, e.g., to the motor, to cause the motor to rotate, which leads to the vibration unit to vibrate. The levels of vibration can be based on the characteristics of the cataract determined by the controller based on the measured force.

FIGS. 30B - 30D show a calibration process for the assessment of the cataract characteristics using the force transducer. In FIG. 30B, the force transducer 3080 can be used to measure the background noise when the phaco tip 3010 is outside of the surgical site, e.g., at an air ambient for example. The measured noise can be used as a reference force, e.g., establishing a base line force. Under this reference force, the controller 3073 can send no vibration signals 3084A to the haptic receiver 3081, indicating that there is no cataract.

In FIG. 30C, the force transducer 3080 can be used to measure the force when the phaco tip 3010 is at a surgical site in which a soft cataract 3087 is known. The measured force can be used as a characteristic force for soft cataract, e.g., establishing a data point for soft cataract characteristics. Under this soft cataract characteristic force, the controller 3073 can send low vibration signals 3084B to the haptic receiver 3081, indicating that the cataract includes a soft material.

In FIG. 30D, the force transducer 3080 can be used to measure the force when the phaco tip 3010 is at a surgical site in which a hard cataract 3088 is known. The measured force can be used as a characteristic field for hard cataract, e.g., establishing a data point for hard cataract characteristics. Under this hard cataract characteristic field, the controller 3073 can send high vibration signals 3084C to the haptic receiver 3081, indicating that the cataract includes a hard material.

After the calibration of measuring the ambient, a soft cataract and a hard cataract, the controller can have data point for relating the measured force to the hardness of the cataract, and can send vibration signals to the haptic receiver according to the assessed characteristics.

In some embodiments, the material haptic feedback can be configured to provide information about the posterior capsule, such as how close the phaco tip is with respect to the posterior capsule. For example, the material haptic feedback can determine the density of the material surrounding the phaco tip, by the ultrasonic haptic feedback or by the force transducer haptic feedback, and the controller can generate the structure of the eye, including the locations of the lens and the posterior capsule, together with the location of the phaco tip. The controller can then provide haptic feedback indicating distances between the phaco tip and the posterior capsule. For example, very far away from the posterior capsule, there is no haptic signal. When the phaco tip is close to, but still at a safe distance from the posterior capsule, such as closer than 6 mm, closer than 5 mm, closer than 4 mm, closer than 3 mm, or closer than 2 mm, the haptic feedback can provide a low level vibration, to alert the surgeon that the posterior capsule is nearby. When the phaco tip is dangerously close to the posterior capsule, such as closer than 3 mm, closer than 2 mm, or closer than 1 mm, the haptic feedback can provide a high level vibration, to alert the surgeon to retract the phaco tip to avoid damaging the posterior capsule.

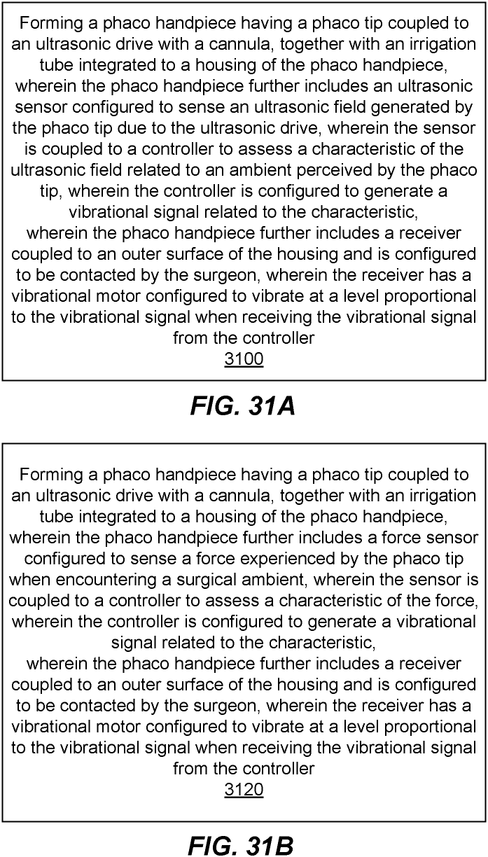

FIGS. 31A - 31B illustrate flow charts for forming a phaco handpiece having a haptic feedback according to some embodiments. In FIG. 31A, operation 3100 forms a phaco handpiece having a phaco tip coupled to an ultrasonic drive with a cannula, together with an irrigation tube integrated to a housing of the phaco handpiece. The phaco handpiece can further include an ultrasonic sensor configured to sense an ultrasonic field generated by the phaco tip due to the ultrasonic drive, with the sensor coupled to a controller to assess a characteristic of the ultrasonic field related to an ambient perceived by the phaco tip, and with the controller configured to generate a vibration signal related to the characteristic.

The phaco handpiece can further include a receiver coupled to an outer surface of the housing and is configured to be contacted by the surgeon, with the receiver having a vibration motor configured to vibrate at a level proportional to the vibration signal when receiving the vibration signal from the controller.

In FIG. 31B, operation 3120 forms a phaco handpiece having a phaco tip coupled to an ultrasonic drive with a cannula, together with an irrigation tube integrated to a housing of the phaco handpiece. The phaco handpiece can further include a force sensor configured to sense a force experienced by the phaco tip when encountering a surgical ambient, with the sensor coupled to a controller to assess a characteristic of the force, and with the controller configured to generate a vibration signal related to the characteristic

The phaco handpiece can further include a receiver coupled to an outer surface of the housing and is configured to be contacted by the surgeon, with the receiver having a vibration motor configured to vibrate at a level proportional to the vibration signal when receiving the vibration signal from the controller.

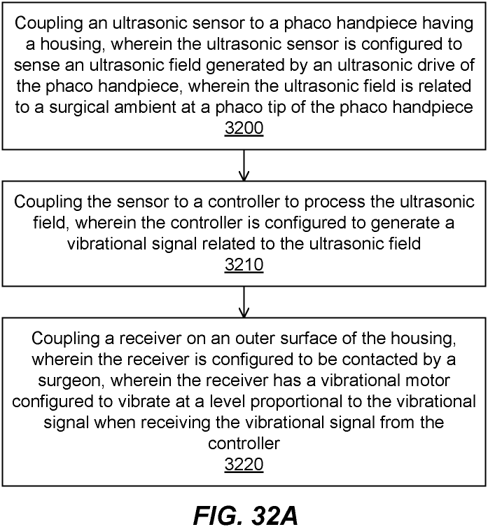

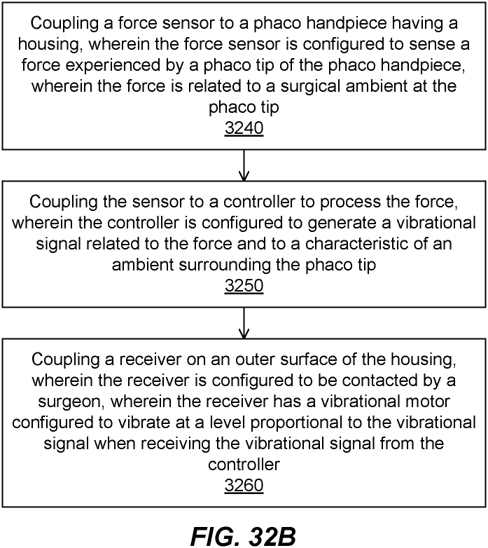

FIGS. 32A - 32B illustrate flow charts for operating a phaco handpiece having a haptic feedback according to some embodiments. In FIG. 32A, operation 3200 couples an ultrasonic sensor to a phaco handpiece having a housing, with the ultrasonic sensor configured to sense an ultrasonic field generated by an ultrasonic drive of the phaco handpiece, and with the ultrasonic field related to a surgical ambient at a phaco tip of the phaco handpiece.

Operation 3210 couples the sensor to a controller to process the ultrasonic field, with the controller configured to generate a vibration signal related to the ultrasonic field. Operation 3220 couples a receiver on an outer surface of the housing, with the receiver configured to be contacted by a surgeon, and with the receiver having a vibration motor configured to vibrate at a level proportional to the vibration signal when receiving the vibration signal from the controller.

In FIG. 32B, operation 3240 couples a force sensor to a phaco handpiece having a housing, with the force sensor configured to sense a force experienced by a phaco tip of the phaco handpiece, and with the force related to a surgical ambient at the phaco tip.

Operation 3250 couples the sensor to a controller to process the force, with the controller configured to generate a vibration signal related to the force and to a characteristic of an ambient surrounding the phaco tip. Operation 3260 couples a receiver on an outer surface of the housing, with the receiver configured to be contacted by a surgeon, and with the receiver having a vibration motor configured to vibrate at a level proportional to the vibration signal when receiving the vibration signal from the controller.

In some embodiments, the present invention discloses a phaco handpiece, e.g., a medical handpiece configured to perform phaco emulsification surgery. The medical handpiece can include a haptic feedback configured to provide a characteristic of an environment surrounding the phaco tip. The haptic feedback can include an ultrasonic sensor configured to sense an ultrasonic field of the environment to provide to a controller.

The haptic feedback can include a vibrationable plate coupled to a vibrating motor, with the controller configured to process the ultrasonic field to generate a control signal to the vibrating motor related to a hardness level of the environment. The vibrationable plate can be disposed on an outer surface of the housing, and configured to let a surgeon holding the medical handpiece to receive a vibration indicating that the hardness level of the environment at the phaco tip.

In some embodiments, the haptic feedback can include a force transducer coupled to the sonotrode and is configured to sense a force encountered by the phaco tip to provide to a controller. The haptic feedback can include a vibrationable plate coupled to a vibrating motor, with the controller configured to process the force to generate a control signal to the vibrating motor related to a hardness level of an environment encountered by the phaco tip. The vibrationable plate can be disposed on an outer surface of the housing, and configured to let a surgeon holding the medical handpiece to receive a vibration indicating that the hardness level of the environment at the phaco tip.

In some embodiments, the medical handpiece can additionally include an on-off haptic feedback configured to provide an operation indication of the medical handpiece, e.g., the medical handpiece can include two haptic feedbacks. One or two vibrating plates can be formed on the housing to provide the on-off haptic feedback and the material haptic feedback

### Multiple Frequency Phaco Handpiece

In some embodiments, the phaco handpiece can be configured to vibrate at different ultrasonic frequencies, e.g., selectable to vibrate at a frequency among multiple possible frequencies. The different frequencies can be selected based on the structure and characteristics of the cataract, for example, selected based on an emulsification power on the cataract material. The ultrasonic transducer can include multiple oscillation circuits that generate ultrasonic vibrations at different frequencies. In addition, a selector can be included at the phaco handpiece to allow the surgeon to switch frequencies during the surgical operation.

Higher ultrasonic frequencies can be more effective in emulsifying hard cataract, e.g., hard nucleus of the crystalline lens at the patient’s eye. Further, higher frequencies can result in shorter surgical time, leading to less stress and low damage on the patient. For example, studies comparing 27.5 kHz and 42 kHz emulsification process show that the emulsification time is significantly higher in the lower frequency process. Raising the ultrasonic frequency can be more advantageous as compared to raising power or duty cycle of the ultrasonic transducer.

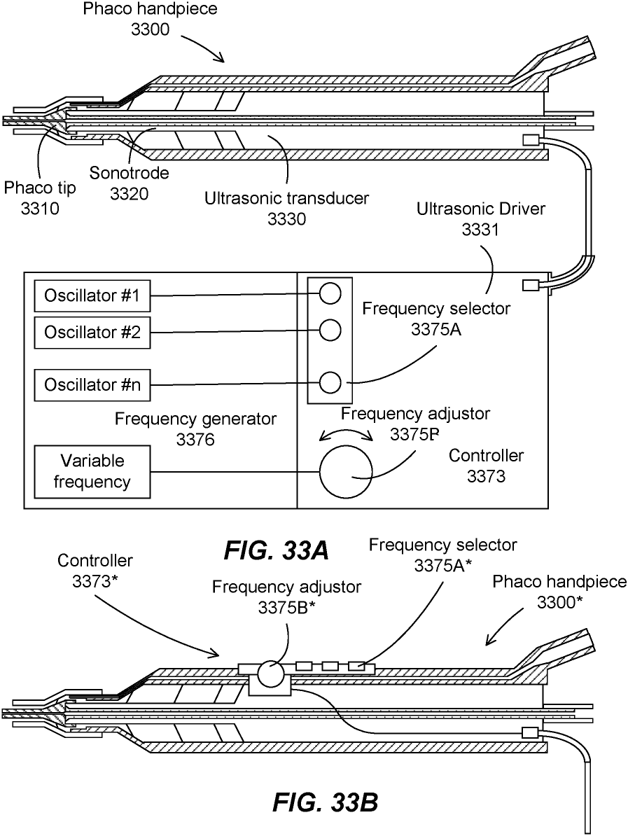

FIGS. 33A - 33B illustrate a phaco handpiece configured for multiple ultrasonic frequencies according to some embodiments. In FIG. 33A, a phaco handpiece 3300 can have an ultrasonic transducer 3330 configured to generate vibrations at an ultrasonic frequency. The vibrations can be amplified by a sonotrode 3320, and then transferred to a phaco tip 3310 for emulsifying cataract materials in an eye site.

The ultrasonic transducer can include piezo elements that can vibrate under the application of a control signal. The piezo elements can vibrate at a frequency of the control signal, thus, by changing the control signal, the ultrasonic transducer can vibrate at a different frequency. The ultrasonic transducer can be coupled to an ultrasonic driver 3331, which can include a frequency generator 3376 and a frequency controller 3373.

In some embodiments, the ultrasonic driver 3331 can include multiple oscillators connected to a frequency selector 3375A to allow a surgeon to discretely selecting a frequency. The surgeon can select a frequency for the phaco handpiece based on the observation and experience of the cataract under the surgery. In some embodiments, the hand piece can has a haptic feedback, such as an ultrasonic haptic feedback or a force feedback described above, to alert the surgeon regarding to the characteristics of the cataract. Based on the cataract characteristics, the surgeon can select appropriate ultrasonic frequency for the emulsification process, such as higher frequency for hard cataract, and lower frequency for soft cataract. As shown, there are multiple frequencies to be selected from. Other configurations can be used, such as 2, 3, or 4 frequencies.

In some embodiments, the ultrasonic driver 3331 can include a variable frequency generator connected to a frequency adjustor 3375B. The frequency adjustor can allow the surgeon to continuously adjust the frequency for the surgery process. As shown, the controller 3373 is located at the ultrasonic driver 3331, e.g., located remotely from the phaco handpiece.

FIG. 33B shows another configuration of a multiple frequency phaco handpiece. A controller 3373* can be formed on the phaco handpiece 3300*, which can allow the surgeon to change the ultrasonic frequency quickly, e.g., during the emulsification process without first stopping to reach the ultrasonic driver for changing frequency.

The phaco handpiece 3300* can include a controller 3373* having the discrete frequency selector 3375A*, the continuous frequency adjustor 3375B*, or both the discrete frequency selector and continuous frequency adjustor.

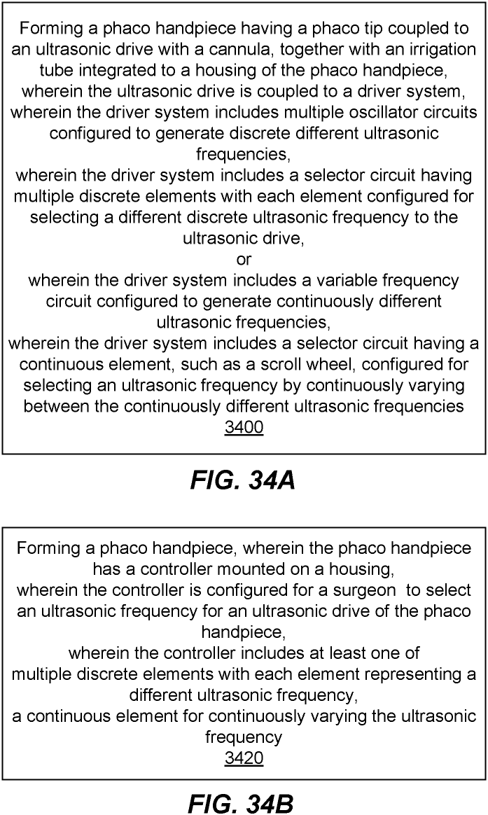

FIGS. 34A - 34B illustrate flow charts for forming a phaco handpiece having multiple frequencies according to some embodiments. In FIG. 34A, operation 3400 forms a phaco handpiece having a phaco tip coupled to an ultrasonic drive with a cannula, together with an irrigation tube integrated to a housing of the phaco handpiece. The ultrasonic drive can be coupled to a driver system.

The driver system can include multiple oscillator circuits configured to generate discrete different ultrasonic frequencies. The driver system can further include a selector circuit having multiple discrete elements with each element configured for selecting a different discrete ultrasonic frequency to the ultrasonic drive.

Alternatively or additionally, the driver system can include a variable frequency circuit configured to generate continuously different ultrasonic frequencies. The driver system can further include a selector circuit having a continuous element, such as a scroll wheel, configured for selecting an ultrasonic frequency by continuously varying between the continuously different ultrasonic frequencies.

In FIG. 34B, operation 3420 forms a phaco handpiece, with the phaco handpiece having a controller mounted on a housing with the controller configured for a surgeon to select an ultrasonic frequency for an ultrasonic drive of the phaco handpiece.

The controller can be configured to include multiple discrete elements with each element representing a different ultrasonic frequency, a continuous element for continuously varying the ultrasonic frequency, or both the multiple discrete elements and the continuous element.

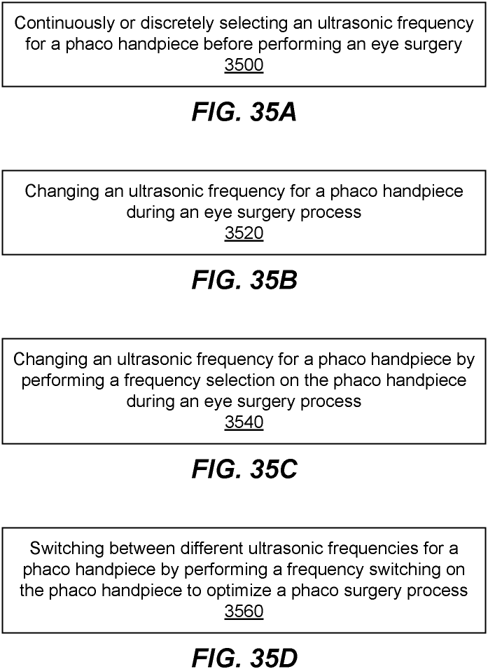

FIGS. 35A - 35D illustrate flow charts for operating a phaco handpiece having multiple frequencies according to some embodiments. In FIG. 35A, operation 3500 continuously or discretely selects an ultrasonic frequency for a phaco handpiece before performing an eye surgery. In FIG. 35B, operation 3520 changes an ultrasonic frequency for a phaco handpiece during an eye surgery process. In FIG. 35C, operation 3540 changes an ultrasonic frequency for a phaco handpiece by performing a frequency selection on the phaco handpiece during an eye surgery process. In FIG. 35D, operation 3560 switches between different ultrasonic frequencies for a phaco handpiece by performing a frequency switching on the phaco handpiece to optimize a phaco surgery process.

In some embodiments, the present invention discloses a phaco handpiece, e.g., a medical handpiece configured to perform phaco emulsification surgery. The medical handpiece can further include a frequency control element configured to select or adjust an ultrasonic frequency generated by the ultrasonic transducer. The frequency control element can include at least one of a discrete frequency selection or a variable frequency selection. The frequency control element can communicate with a controller configured to supply an oscillation signal to the ultrasonic transducer for the ultrasonic transducer to generate the selected ultrasonic frequency.

In some embodiments, the medical handpiece can further include an on-off haptic feedback configured to provide an operation indication of the medical handpiece. The medical handpiece can further include a material haptic feedback configured to provide a characteristic of an environment surrounding the phaco tip. One or two vibrating plates with or without a frequency controller can be formed on the housing to provide the haptic feedbacks and a selection of ultrasonic frequency.

In some embodiments, the haptic feedback and the frequency controller can be combined in the phaco handpiece. The distinction between the different haptic feedbacks can be provided by different amplitudes of vibration, different frequencies of vibration, or different pulse patterns of vibration.

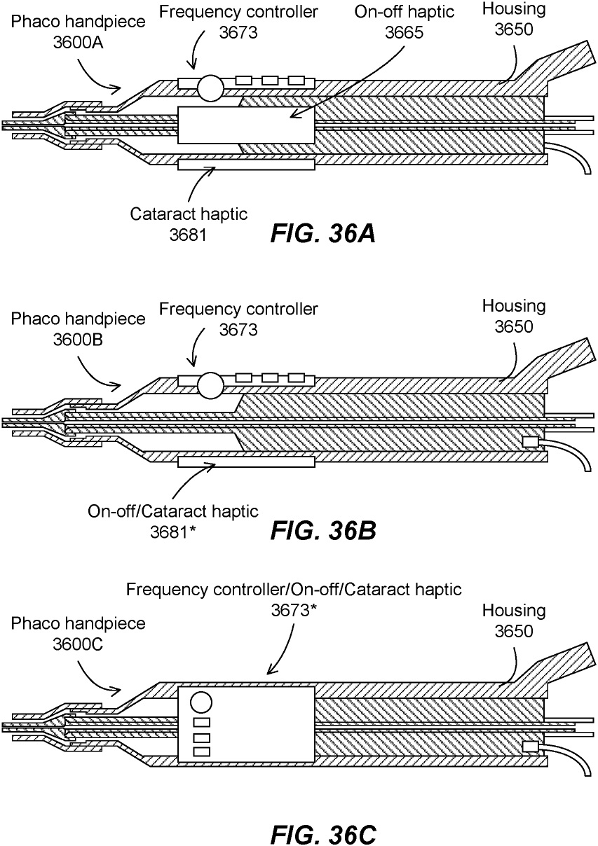

FIGS. 36A - 36C illustrate configurations of phaco handpieces having multiple haptic feedbacks with a frequency controller according to some embodiments. In FIG. 36A, a phaco handpiece 3600A can be configured with an on-off haptic feedback 3665, a material haptic feedback 3681, and a frequency controller 3673, disposed separately on the housing 3650. The on-off haptic feedback can be configured to provide a vibration on a receiver at a finger of the surgeon hand when there are ultrasonic vibrations at the phaco tip of the phaco handpiece. The on-off haptic feedback mechanism can include an ultrasonic sensor or a vibration unit, as discussed above. The material haptic feedback can be configured to provide different vibration patterns, e.g., different amplitudes, different frequencies, or different pulse sequences, to let the surgeon know about the characteristics of the cataract, such as a hard tissue or a soft tissue. The material haptic feedback mechanism can include an ultrasonic sensor or a force sensor, as discussed above. The frequency controller can allow the surgeon to change the frequency of the ultrasonic drive, either by a discrete selection, a continuous selection, or both discrete and continuous selection. As shown, both discrete and continuous selection are included in the phaco handpiece, both only the discrete selection or only the continuous selection can be incorporated in a phaco handpiece.

In FIG. 36B, a phaco handpiece 3600B can be configured with an on-off haptic feedback, a material haptic feedback, and a frequency controller 3673, with the frequency controller being a separate component and the on-off haptic feedback and the material haptic feedback combined to form an integrated on-off/material haptic feedback 3681*. The integrated haptic feedback 3681* can be configured to provide vibration indications of the presence of vibrations at the phaco tip and also the characteristics of the cataract. The distinction between the phaco tip vibrations and the cataract characteristics can be performed by different vibration patterns, e.g., different amplitudes, different frequencies, or different pulse sequences.

In FIG. 36C, a phaco handpiece 3600C can be configured with an on-off haptic feedback, a material haptic feedback, and a frequency controller 3673, with the frequency controller, the on-off haptic feedback, and the material haptic feedback combined to form an integrated frequency controller/on-off/material haptic feedback 3673*. The integrated haptic feedback 3673* can be configured to provide frequency selection, vibration indications of the presence of vibrations at the phaco tip and also the characteristics of the cataract.

Other configurations can be used, such as the frequency controller can be combined with the on-off haptic feedback or the frequency controller can be combined with the material haptic feedback. Further, one, two or all three components of the on-off haptic feedback, the material haptic feedback, and the frequency controller can be formed on the housing of a phaco handpiece. For example, a phaco handpiece can have a material haptic feedback and a frequency controller, disposed separately or combined on a housing of the phaco handpiece. Other configurations can also be used, such as a phaco handpiece having an on-off haptic feedback and a material haptic feedback, disposed as two separate components or disposed as an integrated component on the housing.

In some embodiments, the present invention discloses a phaco handpiece, e.g., a medical handpiece configured to perform phaco emulsification surgery. The phaco handpiece can include an ultrasonic drive comprising a sonotrode coupled to an ultrasonic transducer, a cannula detachably coupled to a channel through the sonotrode and the ultrasonic transducer of the ultrasonic drive, a connecting piece coupled to the protruded second end of the cannula, a phaco tip coupled to the end of the sonotrode, a housing configured to house the ultrasonic drive, and a sleeve coupled to the housing through the opening of the housing.

1. A medical handpiece comprising
an ultrasonic drive comprising a sonotrode coupled to an ultrasonic transducer,
wherein the ultrasonic comprises a channel through the sonotrode and the ultrasonic transducer,
an aspiration tube detachably coupled to the channel,
wherein the aspiration tube comprises a first aspiration passage,
wherein the aspiration tube comprises a first surface at a first end,
wherein the first surface is configured to form a seal with a second surface of the sonotrode,
wherein the aspiration tube comprises a portion at a second end opposite to the first end, wherein the portion protrudes from the ultrasonic drive and configured to be coupled to an aspiration system,
a phaco tip coupled to the sonotrode,
wherein the phaco tip comprises a second aspiration passage,
wherein the phaco tip comprises a third surface configured to be mated with a fourth surface of the aspiration tube to form a fluid communication between the first and second aspiration passages,
wherein the phaco tip is configured so that when coupled with the sonotrode, the phaco tip exerts a force on the aspiration tube for sealing the third surface of the phaco tip with the fourth surface of the aspiration tube,
a housing configured to house the ultrasonic drive,
wherein the housing comprises a first opening comprising a fifth surface,
wherein the phaco tip is configured to protrude from the first opening with the fifth surface forming a seal around a portion of the phaco tip,
wherein the housing comprises an irrigation tube on or under a surface of the housing,
a sleeve coupled to the housing through the opening of the housing,
wherein the sleeve comprises a second opening comprising a sixth surface,
wherein the sixth surface is configured to make a seal with a seventh surface of the housing around the second opening,
wherein the housing comprises an interface for coupling the irrigation tube with the sleeve for supplying an irrigation liquid through the sleeve to an outside ambient.

2. A medical handpiece as in claim 1,
wherein the phaco tip comprises an inner thread configured to be mated with an outer thread of the sonotrode,
wherein the phaco tip is configured so that when threaded with the sonotrode, the phaco tip exerts a force on the aspiration tube for sealing the third surface of the phaco tip with the fourth surface of the aspiration tube.

3. A medical handpiece as in claim 1,
wherein the first surface comprises a flange surface, configured to mate with the second surface comprising a flat surface.

4. A medical handpiece as in claim 1,
wherein the aspiration tube forms a gap with the channel configured to optimize a vibration transfer from the sonotrode to the aspiration tube,
wherein at least one of the aspiration tube or the channel comprises a roughness surface configured to optimize a vibration transfer from the sonotrode to the aspiration tube.

5. A medical handpiece as in claim 1,
wherein the aspiration tube comprises a softer material than the sonotrode material or the phaco tip material.

6. A medical handpiece as in claim 1,
wherein the third surface comprises a convex surface configured to be mated with a flat, convex or concave surface of the fourth surface, or
wherein the fourth surface comprises a convex surface configured to be mated with a flat, convex or concave surface of the third surface.

7. A medical handpiece as in claim 1,
wherein a compressible ring layer is disposed between the third and the fourth surfaces for forming the seal between the third and the fourth surfaces.

8. A medical handpiece as in claim 1,
wherein the phaco tip is integrated with the aspiration tube,
wherein the integrated phaco tip and aspiration tube is configured as a single unit formed by a same material, or
wherein the integrated phaco tip and aspiration tube is configured as a single unit formed by a same material or two different materials with the phaco tip securely coupled to the aspiration tube, with the secured coupling comprising welding, gluing, crimping. friction welding, or press fitting,.

9. A medical handpiece as in claim 1,
wherein the housing comprises support elements coupled to the ultrasonic transducer,
wherein the housing comprises fin elements contacting the sonotrode to provide support to the housing while minimizing vibration transfer from the sonotrode to the housing.

10. A medical handpiece as in claim 1,
wherein the sleeve is configured to fit tightly around a tip of the phaco tip with the tip of the phaco tip protruded from the sleeve,
wherein the sleeve comprises one or more exit ports to deliver the irrigation liquid from the interface with the irrigation tube.

11. A medical handpiece as in claim 1, further comprising
a housing end configured to seal a third opening of the housing,
wherein the third opening is disposed opposite the first opening,
wherein the housing end is configured to also seal a connecting piece for the aspiration tube and a cable for the ultrasonic drive.

12. A medical handpiece as in claim 1, further comprising
a first haptic feedback configured to provide an operation indication of the medical handpiece,
wherein the first haptic feedback comprises a vibrationable plate coupled to a vibration coupler with the vibration coupler coupled to the sonotrode,
wherein the vibrationable plate is disposed on an outer surface of the housing, and configured to let a surgeon holding the medical handpiece to receive a vibration indicating that the medical handpiece is operating.

13. A medical handpiece as in claim 1, further comprising
a first haptic feedback configured to provide an operation indication of the medical handpiece,
wherein the first haptic feedback comprises a vibrationable plate coupled to a vibrating motor,
wherein the vibrating motor is electrically coupled to a power line configured to power the ultrasonic transducer,
wherein the vibrationable plate is disposed on an outer surface of the housing, and configured to let a surgeon holding the medical handpiece to receive a vibration indicating that the medical handpiece is operating.

14. A medical handpiece as in claim 1, further comprising
a first haptic feedback configured to provide an operation indication of the medical handpiece,
wherein the first haptic feedback comprises a vibrationable plate coupled to a vibrating motor,
wherein the first haptic feedback comprises an adjustable component configured to adjust a vibration level of the vibrationable plate based on a comfort level of a surgeon operating the medical handpiece.

15. A medical handpiece as in claim 1, further comprising
a second haptic feedback configured to provide a characteristic of an environment surrounding the phaco tip,
wherein the second haptic feedback comprises an ultrasonic sensor configured to sense an ultrasonic field of the environment to provide to a controller,
wherein the second haptic feedback comprises a vibrationable plate coupled to a vibrating motor,
wherein the controller is configured to process the ultrasonic field to generate a control signal to the vibrating motor related to a hardness level of the environment,
wherein the vibrationable plate is disposed on an outer surface of the housing, and configured to let a surgeon holding the medical handpiece to receive a vibration indicating that the hardness level of the environment at the phaco tip.

16. A medical handpiece as in claim 1, further comprising
a second haptic feedback configured to provide a characteristic of an environment surrounding the phaco tip,
wherein the second haptic feedback comprises a force transducer coupled to the sonotrode and is configured to sense a force encountered by the phaco tip to provide to a controller,
wherein the second haptic feedback comprises a vibrationable plate coupled to a vibrating motor,
wherein the controller is configured to process the force to generate a control signal to the vibrating motor related to a hardness level of an environment encountered by the phaco tip,
wherein the vibrationable plate is disposed on an outer surface of the housing, and configured to let a surgeon holding the medical handpiece to receive a vibration indicating that the hardness level of the environment at the phaco tip.

17. A medical handpiece as in claim 1, further comprising
a second haptic feedback configured to notify about a distance between the phaco tip with a posterior capsule during an eye surgery,
wherein the second haptic feedback comprises an ultrasonic sensor or a force transducer configured to sense a presence of the posterior capsule,
wherein the second haptic feedback comprises a vibrationable plate coupled to a vibrating motor with the vibrationable plate configured to vibrate when the distance between the phaco tip to the posterior capsule is less than a predetermined distance.

18. A medical handpiece as in claim 1, further comprising
a frequency control element configured to select or adjust an ultrasonic frequency generated by the ultrasonic transducer,
wherein the frequency control element comprises at least one of a discrete frequency selection or a variable frequency selection,
wherein the frequency control element is communicated with a controller configured to supply an oscillation signal to the ultrasonic transducer for the ultrasonic transducer to generate the selected ultrasonic frequency.

19. A medical handpiece comprising
an ultrasonic drive comprising a sonotrode coupled to an ultrasonic transducer,
wherein the ultrasonic comprises a channel through the sonotrode and the ultrasonic transducer,
an aspiration tube detachably coupled to the channel,
wherein the aspiration tube comprises a first surface,
wherein the first sealing surface is configured to form a seal with a second surface of the sonotrode,
a phaco tip coupled to the sonotrode,
wherein the phaco tip comprises a third surface configured to be mated with a fourth surface of the aspiration tube,
wherein the phaco tip comprises an inner thread configured to be mated with an outer thread of the sonotrode,
wherein the phaco tip is configured so that when threaded with the sonotrode, the phaco tip exerts a force on the aspiration tube for sealing the third surface of the phaco tip with the fourth surface of the aspiration tube,
a housing configured to house the ultrasonic drive,
wherein the housing comprises a first opening comprising a fifth surface,
wherein the phaco tip is configured to protrude from the first opening with the fifth surface forming a seal around a portion of the phaco tip,
wherein the housing comprises an irrigation tube on or under a surface of the housing,
a sleeve coupled to the housing through the opening of the housing,
wherein the sleeve comprises a second opening comprising a sixth surface,
wherein the sixth surface is configured to make a seal with a seventh surface of the housing around the second opening,
wherein the housing comprises an interface for coupling the irrigation tube with the sleeve for supplying an irrigation liquid through the sleeve to an outside ambient,
a first haptic feedback configured to provide an operation indication of the medical handpiece,
wherein the first haptic feedback comprises a vibrationable plate coupled to a vibration coupler with the vibration coupler coupled to the sonotrode,
wherein the vibrationable plate is disposed on an outer surface of the housing, and configured to let a surgeon holding the medical handpiece to receive a vibration indicating that the medical handpiece is operating.

20. A medical handpiece comprising
an ultrasonic drive comprising a sonotrode coupled to an ultrasonic transducer,
wherein the ultrasonic comprises a channel through the sonotrode and the ultrasonic transducer,
an aspiration tube detachably coupled to the channel,
wherein the aspiration tube comprises a first surface,
wherein the first sealing surface is configured to form a seal with a second surface of the sonotrode,
a phaco tip coupled to the sonotrode,
wherein the phaco tip comprises a third surface configured to be mated with a fourth surface of the aspiration tube,
wherein the phaco tip comprises an inner thread configured to be mated with an outer thread of the sonotrode,
wherein the phaco tip is configured so that when threaded with the sonotrode, the phaco tip exerts a force on the aspiration tube for sealing the third surface of the phaco tip with the fourth surface of the aspiration tube,
a housing configured to house the ultrasonic drive,
wherein the housing comprises a first opening comprising a fifth surface,
wherein the phaco tip is configured to protrude from the first opening with the fifth surface forming a seal around a portion of the phaco tip,
wherein the housing comprises an irrigation tube on or under a surface of the housing,
a sleeve coupled to the housing through the opening of the housing,
wherein the sleeve comprises a second opening comprising a sixth surface,
wherein the sixth surface is configured to make a seal with a seventh surface of the housing around the second opening,
wherein the housing comprises an interface for coupling the irrigation tube with the sleeve for supplying an irrigation liquid through the sleeve to an outside ambient,
a first haptic feedback configured to provide an operation indication of the medical handpiece,
wherein the first haptic feedback comprises a first vibrationable plate coupled to a vibration coupler with the vibration coupler coupled to the sonotrode,
wherein the first vibrationable plate is disposed on an outer surface of the housing, and configured to let a surgeon holding the medical handpiece to receive a first vibration indicating that the medical handpiece is operating,
a second haptic feedback configured to provide a presence of a posterior capsule in an eye surgery,
wherein the second haptic feedback comprises a force transducer coupled to the sonotrode and is configured to sense a force encountered by the phaco tip to provide to a controller,
wherein the second haptic feedback comprises a second vibrationable plate coupled to a vibrating motor,
wherein the controller is configured to process the force to generate a control signal to the vibrating motor related to a hardness level of an environment encountered by the phaco tip,
wherein the second vibrationable plate is disposed on an outer surface of the housing, and configured to let a surgeon holding the medical handpiece to receive a second vibration indicating a distance between the phaco tip and the posterior capsule,
a frequency control element configured to select or adjust an ultrasonic frequency generated by the ultrasonic transducer,
wherein the frequency control element comprises at least one of a discrete frequency selection or a variable frequency selection,
wherein the frequency control element is communicated with a controller configured to supply an oscillation signal to the ultrasonic transducer for the ultrasonic transducer to generate the selected ultrasonic frequency.
